(19) United States
(12) Patent Application Publication
(10) Pub. No.: US 2026/0048269 A1
(43) Pub. Date: Feb. 19, 2026

(54) DETECTING ALIGNMENT OF AN EXTERNAL CHARGER TO AN IMPLANTABLE MEDICAL DEVICE

(71) Applicant: Boston Scientific Scimed, Inc., Maple Grove, MN (US)

(72) Inventors: Joey Chen, Stevenson Ranch, CA (US); Jonathan Philip Larcom, Simi Valley, CA (US); Vuong Tuan Nguyen, Glendale, CA (US); Keith R. Maile, New Brighton, MN (US); Jason Lahr, Coon Rapids, MN (US); Kevin James Ivanca, Minneapolis, MN (US)

(21) Appl. No.: 19/296,379

(22) Filed: Aug. 11, 2025

Related U.S. Application Data
(60) Provisional application No. 63/682,999, filed on Aug. 14, 2024.

(51) Int. Cl. A61N 1/378; G01B 7/31; H02J 50/10; H02J 50/90

(52) U.S. Cl. CPC A61N 1/3787; G01B 7/31; H02J 50/10; H02J 50/90; H02J 2207/20

(57) ABSTRACT

According to an aspect, an external charger may include a power converter configured to generate a magnetic field on a transmitter member of the external charger. An external charger may include an alignment detector configured to: detect a first amplitude on an inner sensor member of the external charger, detect a second amplitude on an outer sensor member of the external charger, and compute an alignment value based on the first amplitude and the second amplitude, the alignment value representing a degree of alignment between the transmitter member and a receiver member of the implantable medical device. An external charger may include an indicator generator configured to generate an indicator based on the alignment value.

### CROSS-REFERENCE TO RELATED APPLICATION

This application claims priority to U.S. Provisional Patent Application No. 63/682,999, filed on Aug. 14, 2024, entitled “DETECTING ALIGNMENT OF AN EXTERNAL CHARGER TO AN IMPLANTABLE MEDICAL DEVICE”, the disclosure of which is incorporated by reference herein in its entirety.

### TECHNICAL FIELD

This disclosure relates generally to detecting an alignment of an external charger with an implantable medical device.

### BACKGROUND

A medical device may be implanted into the body of a patient, and the medical device may have electronic circuitry powered by a battery source that is rechargeable. To recharge the battery source, a transmitter member of the external charger (e.g., outside of the body) may be aligned with a receiver member of the medical device (e.g., inside of the body). Some conventional approaches for detecting alignment between an external charger and an implantable medical device may be relatively complex involving a high level of processing power.

### SUMMARY

The techniques described herein relate to an alignment detector configured to detect an alignment between the external charger and the implantable medical device to improve wireless power transfer. The alignment detector computes an alignment value that represents the degree of positioning between a receiver member (e.g., a receiver coil) of an implantable medical device and a transmitter member (e.g., a transmitter coil or charge coil) of the external charger, and an indicator generator may provide an alignment indicator (e.g., a visual or audio indicator) to the user based on the alignment value. The external charger includes an inner sensor member (e.g., an inner sense coil) and an outer sensor member (e.g., an outer sense coil). The alignment detector computes the alignment value based on a ratio of an amplitude on the inner sensor member and an amplitude on the outer sensor member. In some examples, the ratio is referred to as a sense coil amplitude ratio. In some examples, the ratio between the outer and inner members increases as the transmitter member becomes closer and/or centered with respect to the receiver member. In some examples, the transmitted power can be variable, and the ratio is independent of the transmitted power.

Implementations can include one or more of the following features, alone, or in any combination with each other.

In some aspects, the techniques described herein relate to a method for aligning an external charger and an implantable medical device, the method including: generating a magnetic field in a transmitter member of the external charger; detecting a first amplitude on an inner sensor member of the external charger; detecting a second amplitude on an outer sensor member of the external charger; computing an alignment value based on the first amplitude and the second amplitude, the alignment value representing a degree of alignment between the transmitter member and a receiver member of the implantable medical device; and generating an indicator based on the alignment value.

In some aspects, the techniques described herein relate to an external charger for an implantable medical device, the external charger including: a power converter configured to generate a magnetic field on a transmitter member of the external charger; an alignment detector configured to: detect a first amplitude on an inner sensor member of the external charger; detect a second amplitude on an outer sensor member of the external charger; and compute an alignment value based on the first amplitude and the second amplitude, the alignment value representing a degree of alignment between the transmitter member and a receiver member of the implantable medical device; and an indicator generator configured to generate an indicator based on the alignment value.

In some aspects, the techniques described herein relate to an apparatus including: an implantable medical device including a receiver member; and an external charger having a transmitter member, an inner sensor member, and an outer sensor member, the external charger configured to: generate a magnetic field on the transmitter member; detect a first amplitude on the inner sensor member; detect a second amplitude on the outer sensor member; and compute an alignment value based on the first amplitude and the second amplitude, the alignment value representing a degree of alignment between the transmitter member and the receiver member; and generate an indicator based on the alignment value.

### BRIEF DESCRIPTION OF THE DRAWINGS

FIG. 1A is a block diagram of a medical device and a power transmission device with an alignment detector for detecting an alignment value between the medical device and the power transmission device.

FIG. 1B illustrates an example of a power transmission device.

FIG. 1C illustrates a graph depicting values of a coil ratio for increasing distance between the medical device and the power transmission device.

FIG. 1D illustrates a graph depicting increasing values of a coil ratio for increasing values of an inner sense voltage according to an aspect.

FIG. 1E illustrates an example of transmitting load-shift keying (LSK) data based on voltage and/or current readings in a medical device according to an aspect.

FIG. 1F illustrates an example of LSK data indicating an alignment level with an external charger according to an aspect.

FIG. 2A illustrates a system including an example implantable fluid-operated inflatable device according to an aspect.

FIG. 2B illustrates an example of an artificial urinary sphincter device according to an aspect.

FIG. 3 illustrates a flowchart depicting example operations for generating an alignment value between a medical device and a power transmission device.

FIG. 4A is an exploded view of an example valve device of a fluid control system of a fluid-operated inflatable device.

FIG. 4B is another exploded view of the example valve device shown in FIG. 4A.

FIG. 4C is a cross-sectional view of the example valve device shown in FIG. 4A, in a closed position.

FIG. 4D is a cross-sectional view of the example valve device shown in FIG. 4A, in an open position.

FIG. 5A is a schematic view of an example valve device including an example auxiliary flow control device, with the example valve device in an open position.

FIG. 5B is a schematic view of an example valve device including an example auxiliary flow control device, with the example valve device in a closed position.

FIG. 6A is an exploded view of an example pump device of a fluid control system of a fluid-operated inflatable device.

FIG. 6B is a cross-sectional view of the example pump device shown in FIG. 6A, in an open position.

FIGS. 7A, 7B, and 7C are cross-sectional views of example pump devices that include a filter for capturing particulate matter in the fluid flow and/or for blocking the particulate matter from entering certain parts of the fluidic system (e.g., for blocking particulate matter from entering a pump chamber of the device).

FIG. 8 is a schematic end view of a filter foil.

FIGS. 9A, 9B, and 9C are cross-sectional views of example pump devices that include a filter for capturing particulate matter in the fluid flow and/or for blocking the particulate matter from entering certain parts of the fluidic system (e.g., for blocking particulate matter from entering a pump chamber of the device).

FIG. 10 is a cross-sectional view of the valve device of FIGS. 5A and 5B, but also including a filter located at an end of a second fluid passageway and a filter located within a first fluid passageway.

FIGS. 11A and 11B show different examples of implantable neurostimulators that can be charged by the disclosed power transmission device.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants and/or external chargers configured to wireless charge the bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

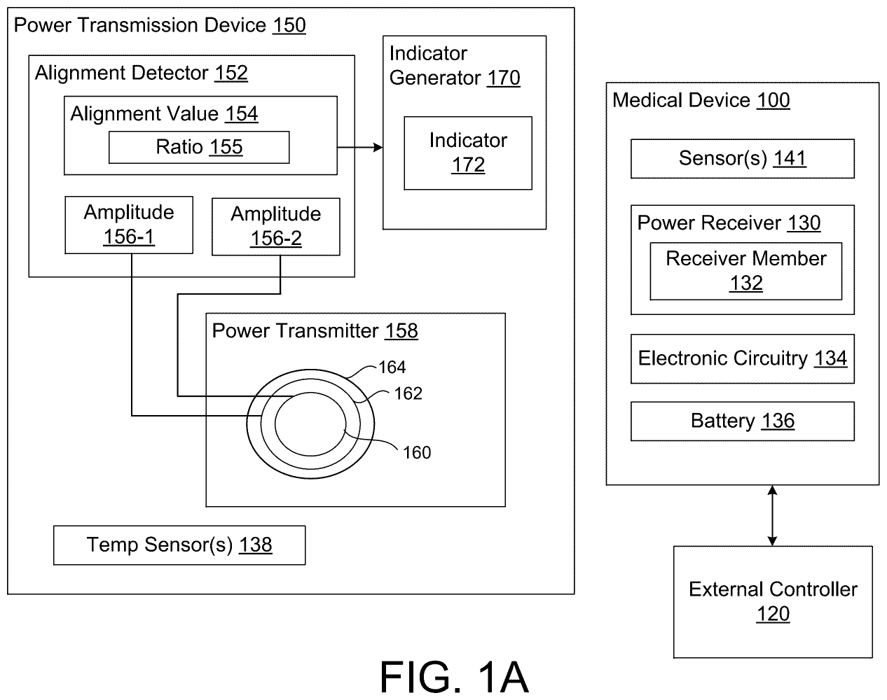

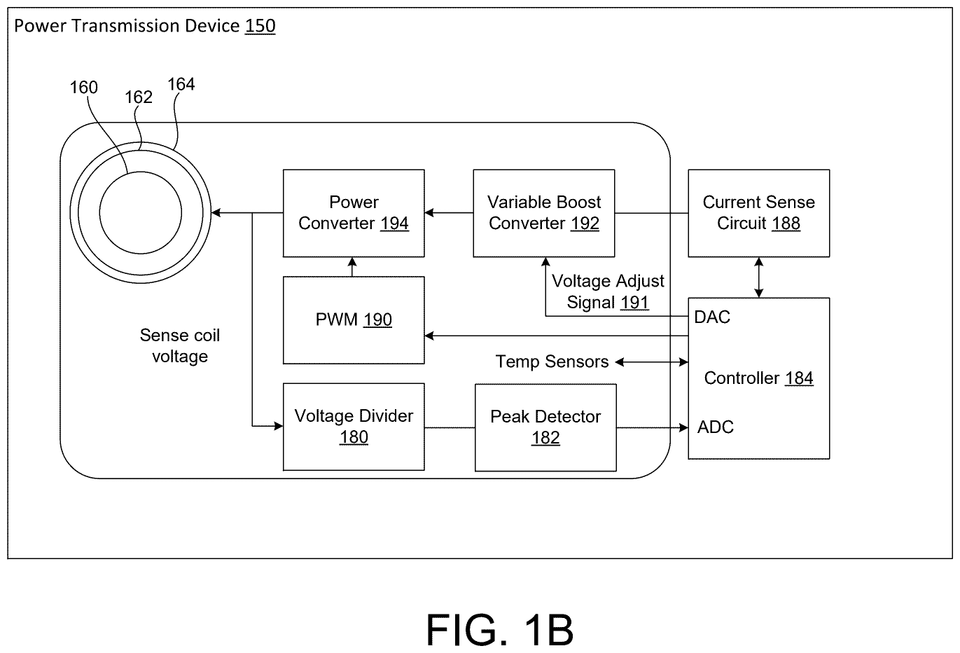

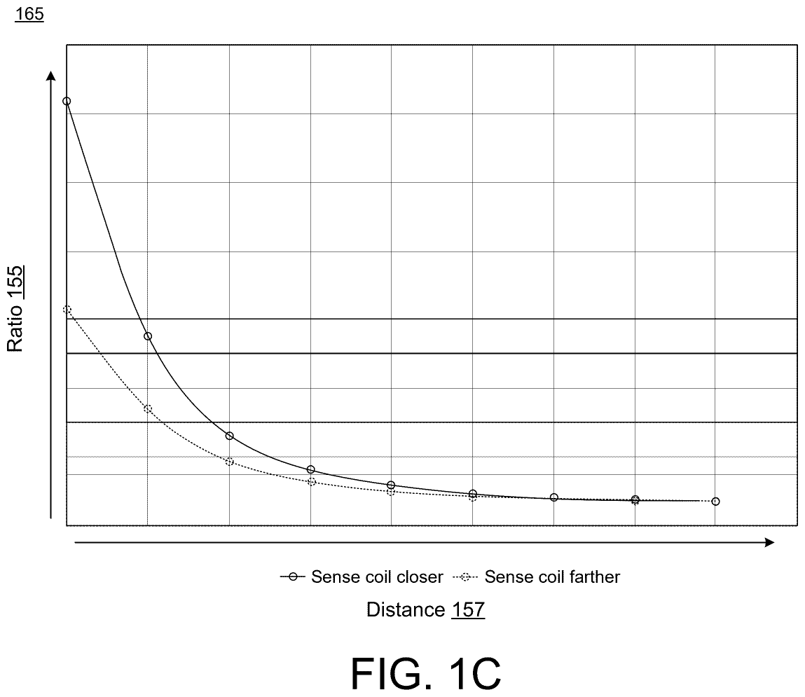

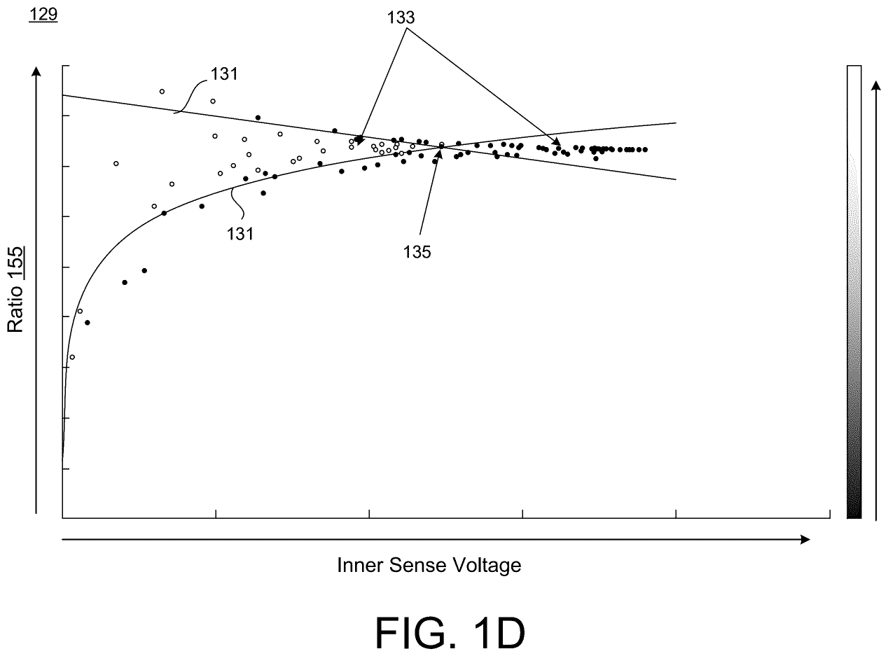

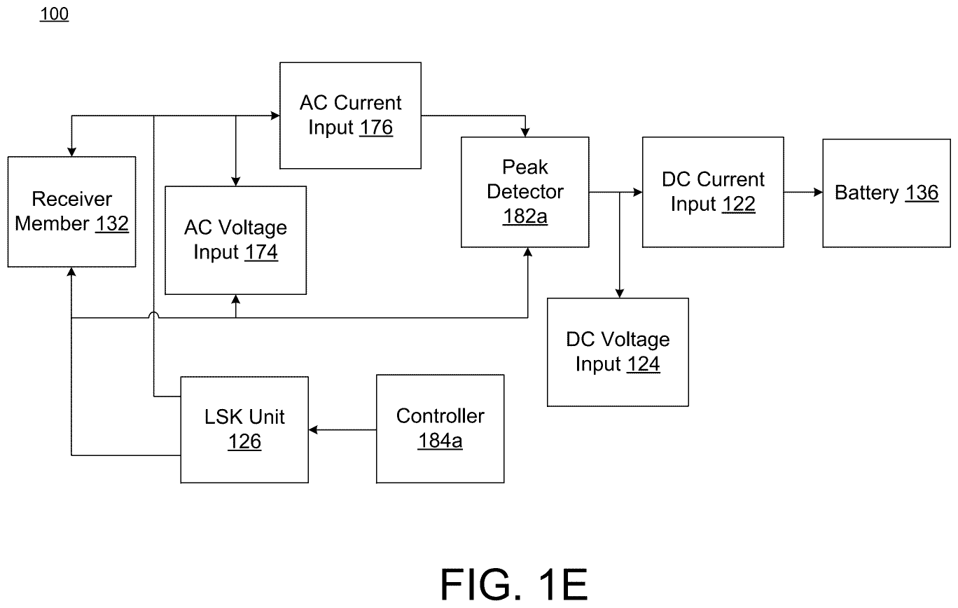

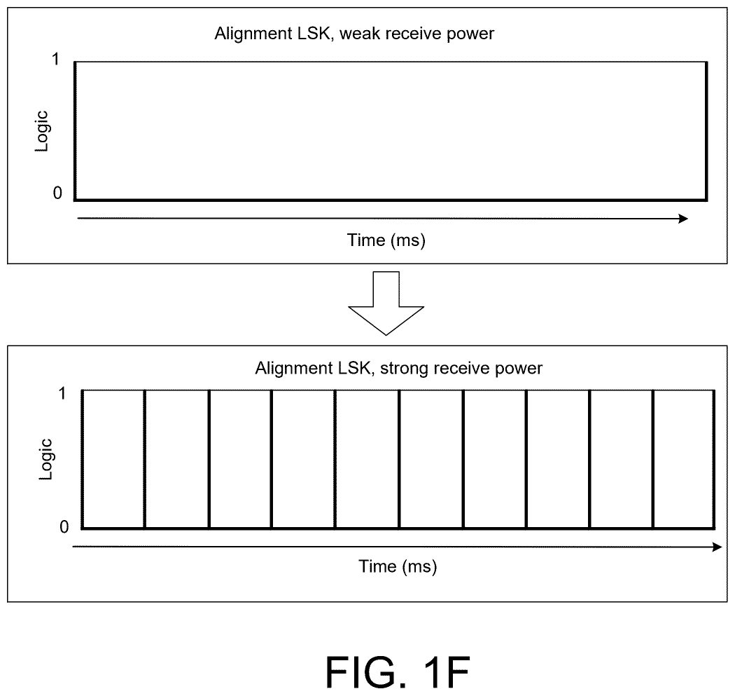

FIG. 1A is a block diagram of an implantable medical device 100 and a power transmission device 150. FIG. 1B illustrates an example of the power transmission device. The medical device 100 can be implanted into the body of a patient. The power transmission device 150 wirelessly charges (e.g., transfers energy to) the medical device 100 while the medical device 100 is implanted in the body of the patient. The transferred energy may charge a battery 136 of the medical device 100 and/or power electronic circuitry 134 of the medical device 100. In some examples, the power transmission device 150 is referred to as an external charger. In some examples, the power transmission device 150 is referred to as a wireless charger (e.g., power is delivered to the medical device 100 without the use of wires or cords between the medical device 100 and the power transmission device 150).

The power transmission device 150 includes a power transmitter 158 configured to emit a magnetic field to wirelessly transfer energy to a power receiver 130 of the medical device 100. In some examples, the power transmitter 158 is referred to as a charging pad (e.g., an external charging pad). The power transmitter 158 includes a transmitter member 164. In some examples, the transmitter member 164 is referred to as a transmitter coil or a charge coil. The power receiver 130 may include a receiver member 132. In some examples, the receiver member 132 is referred to as a receiver coil. The transmitter member 164 emits a magnetic field that propagates outwardly from the transmitter member 164, and, when the receiver member 132 comes within range and in close proximity, the changing magnetic fields induce a voltage in the receiver member 132. In some examples, the transmitter member 164 may be a spiral pattern (e.g., a flat spiral pattern). For increased power transfer performance, the transmitter member 164 and the receiver member 132 may be positioned within a threshold distance and aligned.

In some examples, alignment of the transmitter member 164 and the receiver member 132 may refer the physical positioning of the transmitter member 164 to the receiver member 132 such as a first axis that extends through a center of the transmitter member 164 being within a threshold distance of a second axis that extends through a center of the receiver member 132. In some examples, the alignment of the transmitter member 164 and the receiver member 132 may refer to the strength of the magnetic field coupling such as the amount of magnetic field lines generated by the transmitter member 164 that pass through the receiver member 132. In some examples, the more magnetic field lines that are intercepted by the receiver member 132, the stronger the coupling and the more efficient the power transfer.

The power transmitter 158 includes an inner sensor member 160 and an outer sensor member 162. In some examples, the inner sensor member 160 is referred to as an inner sense coil. In some examples, the outer sensor member 162 is referred to as an outer sense coil. In some examples, the outer sensor member 162 is a size (e.g., a diameter) that is less than a size (e.g., diameter) of the transmitter member 164. The inner sensor member 160 may have a diameter that is smaller than the diameter of the outer sensor member 162.

In some examples, the inner sensor member 160 is a first printed circuit board (PCB) trace, and the outer sensor member 162 is a second PCB trace. In some examples, each of the inner sensor member 160 and the outer sensor member 162 may detect a magnetic field and/or a back electromotive force (EMF) caused by eddy currents, which cancels a portion of the magnetic field in a center portion (e.g., the center) of the power transmitter 158. For example, when the medical device 100 is placed within the range of the transmitter field (e.g., the magnetic field) of the power transmitter 158, the changing magnetic field can induce eddy currents.

In some examples, the inner sensor member 160 includes a circular (conductive) portion. In some examples, the outer sensor member 162 includes a circular (conductive) portion. In some examples, the inner sensor member 160 includes a spiral circular pattern with a flat shape. In some examples, the outer sensor member 162 includes a spiral circular pattern with a flat shape. In some examples, the inner sensor member 160 and/or the outer sensor member 162 may have the same shape and/or structure. In some examples, the inner sensor member 160 and/or the outer sensor member 162 may have different shapes and/or structures. In some examples, the inner sensor member 160 and/or the outer sensor member 162 may have a cloverleaf shape. In some examples, the inner sensor member 160 and/or the outer sensor member 162 may have a butterfly shape.

The power transmission device 150 includes an alignment detector 152 configured to generate an alignment value 154. The alignment value 154 may represent the degree of alignment between the receiver member 132 and the transmitter member 164. In some examples, the transmitter member 164 is referred to as a charge coil or a charging coil. In some examples, the alignment value 154 includes a ratio 155 of an amplitude 156-1 of the inner sensor member 160 and an amplitude 156-2 of the outer sensor member 162. In some examples, the ratio 155 is referred to as a sense amplitude ratio (e.g., a sense coil amplitude ratio). In some examples, the ratio 155 increases as the transmitter member 164 becomes closer and/or centered with respect to the receiver member 132.

The alignment detector 152 may sense (e.g., detect) an amplitude 156-1 of the voltage on the inner sensor member 160 and may sense (e.g., detect) an amplitude 156-2 of the voltage on the outer sensor member 162. In some examples, the alignment detector 152 detects the amplitude 156-1 and the amplitude 156-2 according to a sampling rate. In some examples, by using the amplitude 156-1 and the amplitude 156-2, a lower sampling rate can be used (as compared to some conventional approaches), thereby reducing the amount of processing power. The alignment detector 152 may compute the alignment value 154 as the ratio 155 of the amplitude 156-1 and the amplitude 156-2. In some examples, the ratio 155 between the amplitude 156-1 and the amplitude 156-2 increases as the charge coil (e.g., the transmitter member 164) gets closer and/or better centered with respect to the receiver member 132.

In some examples, the power transmission device 150 includes an indicator generator 170 configured to generate an indicator 172 according to the alignment value 154. The indicator 172 may provide feedback to the user about the alignment of the power transmission device 150 to the medical device 100. In some examples, the indicator 172 is a visual indicator. In some examples, the indicator 172 is an audio indicator. In some examples, the indicator 172 includes a visual and audio indicator. The indicator 172 may change according to the alignment values 154 so that the user can determine whether movement of the power transmission device 150 causes the power transmitter 158 to be better aligned with the power receiver 130 of the medical device 100.

In some examples, referring to FIG. 1B, the power transmission device 150 may include a voltage divider 180, a peak detector 182, and a controller 184. In some examples, the alignment detector 152 includes the voltage divider 180, the peak detector 182, and the controller 184. The voltage divider 180 may include two resistors connected in series across the inner sensor member 160 and the outer sensor member 162.

In some examples, the peak detector 182 may rectify the sense voltages (e.g., sense coil voltages). The peak detector 182 may convert the voltage signal to a voltage waveform signal (e.g., with an envelope level or a peak level). The peak detector 182 receives a first sense coil voltage at the inner sensor member 160 and a second sense coil voltage at the outer sensor member 162. The peak detector 182 may generate a first voltage waveform based on the first sense coil voltage and a second voltage waveform based on the second sense coil voltage. In some examples, the first voltage waveform is an analog voltage signal that includes the peak amplitude at the inner sensor member 160. In some examples, the second voltage waveform is an analog voltage signal that includes the peak amplitude at the outer sensor member 162.

In some examples, the first voltage waveform may represent an envelope signal at the inner sensor member 160. In some examples, the second voltage waveform may represent an envelope signal at the outer sensor member 162. In some examples, the first voltage waveform may represent a rectified signal at the inner sensor member 160. In some examples, the second voltage waveform may represent a rectified signal at the outer sensor member 162.

The peak detector 182 may include one or more diodes. In some examples, the peak detector 182 may include one or more capacitors. In some examples, the peak detector 182 may include one or more diodes and one or more capacitors. In some examples, the peak detector 182 includes a half-wave rectifier circuit (e.g., a single diode). In some examples, the peak detector 182 includes a full-wave rectifier (e.g., four or more diodes arranged in a bridge circuit).

In some examples, the controller 184 includes a microcontroller. In some examples, the controller 184 includes an integrated circuit. In some examples, the controller 184 includes an analog-to-digital converter and/or a digital-to-analog converter. In some examples, the controller 184 includes a memory for storing program code and/or static memory for data storage. In some examples, the controller 184 includes one or more interfaces configured to communicate with sensors (e.g., temperature sensor 138) and/or other components such as a current sense circuit 188.

In some examples, the controller 184 may sample the first and second voltage waveforms according to a sample rate and generate, for each sample, a first digital value representing the amplitude 156-1 at the inner sensor member 160 and a second digital value representing the amplitude 156-2 at the outer sensor member 162. The controller 184 may compute a ratio (e.g., a ratio 155) of the first digital value representing the amplitude 156-1 at the inner sensor member 160 and the second digital value representing the amplitude 156-2 at the outer sensor member 162. In some examples, the controller 184 may store the ratio 155 for each sample (or a portion of the samples). In some examples, once rectified, the sense coil amplitudes (e.g., amplitude 156-1, amplitude 156-2) may be read by the controller 184 at a sample rate (e.g., less than 100 times per second, less than 50 times per second, less than 15 times per second, etc.).

In some examples, the controller 184 receives a temperature of the power transmitter 158 (e.g., a temperature of the transmitter member 164) from a temperature sensor 138 on the power transmission device 150. In some examples, the controller 184 receives the temperature via an inter-integrated circuit (I2C) transmission protocol. In some examples, the controller 184 may adjust the power (e.g., voltage, current) transmitted via the power transmitter 158 based on the temperature sensor 138. In some examples, in response to the temperature being greater than a threshold level, the controller 184 may reduce or stop the transmission of energy via the power transmitter 158.

In some examples, the power transmission device 150 includes a current sense circuit 188 configured to detect an output voltage of the transmitter member 164. In some examples, the current sense circuit 188 may detect an analog voltage signal proportional to a voltage source (e.g., VBAT). In some examples, the power transmission device 150 includes a variable boost converter 192 configured to adjust the voltage from the voltage source according to a voltage adjust signal 191 (e.g., an analog control signal) from the controller 184. In some examples, the variable boost converter 192 includes an analog-to-analog voltage converter. The controller 184 may generate an analog control signal (e.g., a voltage adjust signal 191) using a digital-to-analog converter, and the variable boost converter 192 may increase (or decrease) the voltage according to the voltage adjust signal 191. In some examples, the voltage adjust signal 191 is referred to as a reference signal.

In some examples, the power transmission device 150 includes a pulse width modulator (PWM) 190 configured to control a duty signal based on a control signal from the controller 184. The PWM 190 may output a control signal that switches between a high state and a low state at a fixed frequency. In some examples, the PWM 190 includes a high-resolution PWM. The power transmission device 150 may include a power converter 194 that receives the voltage from the variable boost converter 192 and generates a voltage that is applied to the transmitter member 164 according to the control signal generated by the PWM 190. In some examples, the power converter 194 includes a half-bride circuit. In some examples, the power converter 194 may include switching elements (e.g., transistors) (e.g., Metal-Oxide-Semiconductor Field-Effect Transistors (MOSFETs), configured in a bridge arrangement. These transistors may operate as high-speed electronic switches that can be turned on and off.

In some examples, the medical device 100 includes one or more sensors 141. In some examples, the sensor(s) 141 includes a magnetic field sensor (e.g., a Hall effect sensor). In some examples, the transmitter member 164 includes a magnet. In some examples, the transmitter member 164 includes a static magnet. In some examples the sensor 141 (e.g., the magnetic field sensor) (e.g., Hall effect sensor) may sense the strength of the magnetic field of the static magnet in the transmitter member 164. The strength of the magnetic field may be an indicator (e.g., the indicator 172) of the alignment of the transmitter member 164 and the receiver member 132. In some examples, the strength of the magnetic field may be communicated from the medical device 100 to the user.

In some examples, the electronic circuitry 134 may include a telemetry communication engine (e.g., LSK unit 126) configured to communicate the sensed magnitude strength (e.g., the indicator 172) to an external controller 120, which can display visual feedback of the alignment between the transmitter member 164 and the receiver member 132. The external controller 120 may be a device that can control one or more functions of the medical device 100. In some examples, the medical device 100 communicates the indicator 172 to another device besides an external controller 120 such as a user device (e.g., an application on a smartphone, a computer, or other computing device).

In some examples, the sensor(s) 141 may include one or more temperature sensors that measure the temperature of the medical device 100 (or a portion thereof). In some examples, the temperature sensor is located in an electronic pump of the medical device 100. The alignment detector 152 may use the temperature data of the temperature sensor to generate an indicator 172 about the alignment of the transmitter member 164 with the receiver member 132. Charging will heat the medical device 100. If the transmitter member 164 and the receiver member 132 are out of alignment, the temperature data may indicate uneven temperature data.

In some examples, power transmission device 150 includes an inertial sensor such as a gyroscopic, an accelerometer, and/or an inertial measurement unit (IMU). The inertial sensor may determine a positional data of the power transmission device 150 and use that positional data to determine its location relative to the receiver member 132. The power transmission device 150 may determine an indicator 172 based on the positional data.

FIG. 1C illustrates a graph 165 depicting a ratio 155 for increasing values of a distance 157 between the transmitter member 164 (e.g., transmitter coil) and the receiver member 132 (e.g., receiver coil). In some examples, the ratio 155 between the outer sensor member 162 and the inner sensor member 160 increases as the transmitter member becomes closer and/or centered to the receiver member 132. In some examples, the transmitted power can be variable, and the ratio 155 is independent of the transmitted power of the transmitter member 164.

FIG. 1D illustrates a graph 129 depicting increasing values of a coil ratio 155 for increasing values of an inner sense voltage according to an aspect. In some examples, the alignment detector 152 may define an alignment area 133. The alignment area 133 may be defined by one or more alignment boundaries determined by one or more curves 131 from a curve-fitting algorithm. The curves 131 determined by the curve-fitting algorithm may fit (e.g., best fit) of a two-dimensional (2D) map of ratios 155 versus an inner sense voltage. In some examples, to determine alignment of the external charger, the alignment detector 152 may use the ratio 155 and the inner sensor voltage to determine whether that data point falls within the alignment area 133. The closer the data point becomes to an intersection 135 of the curves 131, the better alignment of transmitter member 164 with the receiver member 132.

FIGS. 1E and 1F illustrate an example of transmitting load-shift keying (LSK) data based on voltage and/or current readings in a medical device according to an aspect. FIG. 1F illustrates an alignment LSK for weak to strong received power. In some examples, instead of the power transmission device 150 computing an indicator 172 that represents a level of alignment between the power transmission device 150 and the medical device 100, the medical device 100 may detect voltage and/or current readings in the receiver member 132, and use those readings as an indicator 172, which are communicated back to the user using LSK. In some examples, the medical device 100 may measure input voltage and/or current to determine the power received by an external charger (e.g., can be either AC or DC measurements). The medical device 100 may transmit backscatter LSK data to indicate an optimal alignment. In some examples, the pulse width could change to indicate alignment. In some examples, the medical device 100 may transmit the backscatter LSK data with the determined power to the external charger.

For example, the medical device 100 may detect an AC voltage input 174 and/or AC current input 176 of the receiver member 132. In some examples, the medical device 100 includes a peak detector 182a configured to compute a DC current input 122 and/or a DC voltage input 124. In some examples, the DC current input 122 is provided to the battery 136. In some examples, a controller 184a on the medical device may compute the power from the measured DC current input 122 and/or the DC voltage input 124 and provide the calculated power to a LSK unit 126 that computes LSK data with the computed power, which can be transmitted by the receiver member 132. In some examples, the LSK unit 126 encodes the detected power and transmits the data to another device, which may be the external charger.

In some examples, the medical device 100 is an implantable fluid-operated inflatable device, which may include a fluid reservoir, an inflatable member, and an electronic control system.

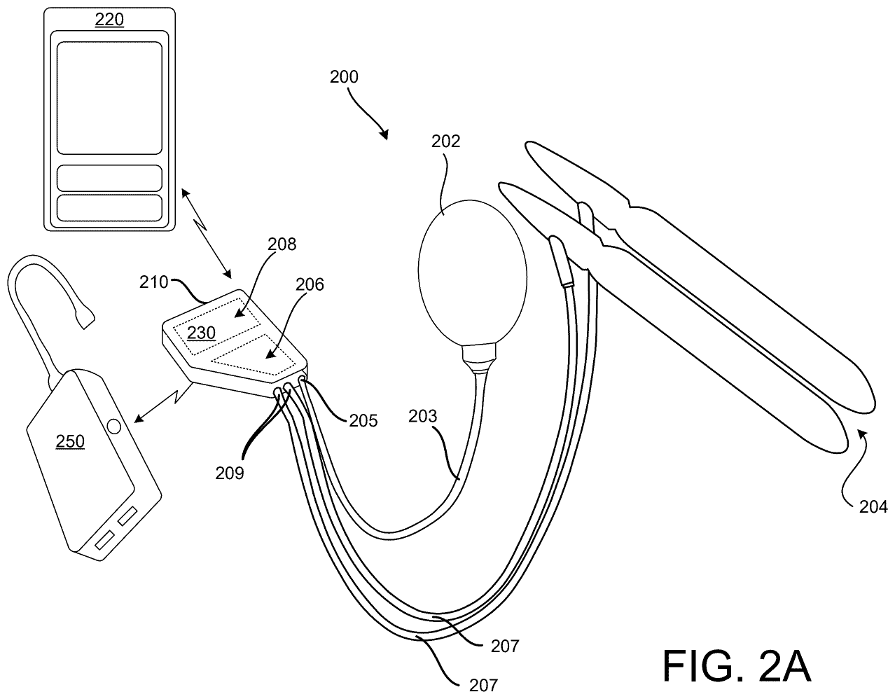

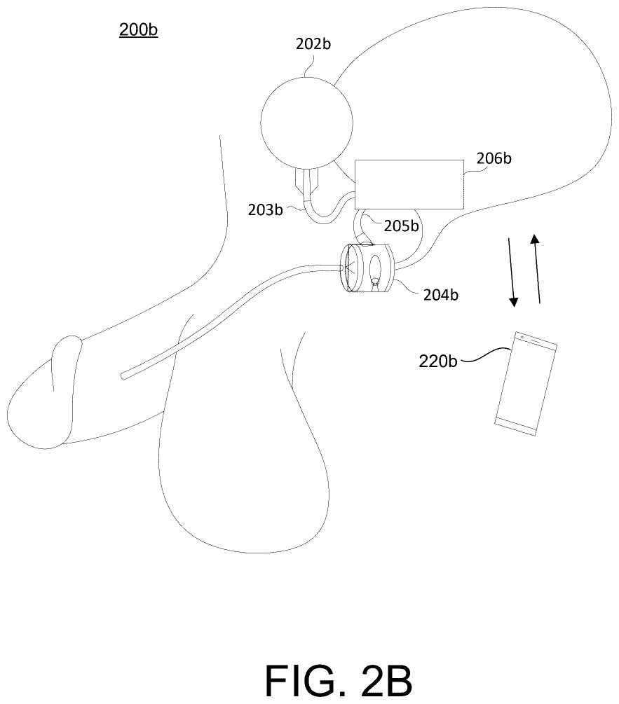

FIG. 2A illustrates an example implantable fluid-operated inflatable device 200 in the form of an example inflatable penile prosthesis. The example inflatable device 200 may be an example of the medical device 100 of FIGS. 1A to IF and may include any of the details discussed with reference to those figures. In some examples, the example implantable fluid-operated inflatable device 200 may be charged using the power transmission device 150 of FIGS. 1A to IF. In some examples, the power receiver 130 (including the receiver member 132), the electronic circuitry 134, battery 136, and/or the sensor(s) 141 are included in the fluid control system 206.

The example inflatable device 200 includes a fluid control system 206 including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 configured to provide for the transfer of fluid between a reservoir 202 and an inflatable member 204 via the fluidics components. In the example shown in FIG. 2A, the inflatable member 204 is in the form of a pair of inflatable cylinders. In the example shown in FIG. 2, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the example shown in FIG. 2A, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220, via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 can be charged and/or recharged by a power transmission device (e.g., the power transmission device 150 of FIGS. 1A to 1F) of the external controller 220, and/or by a power transmission device 250 (e.g., the power transmission device 150 of FIGS. 1A to 1F), that is separate from the external controller 220.

The example implantable fluid-operated inflatable device 200 shown in FIG. 2A includes an electronic control system 208 to provide for control of the operation of the respective inflatable members 204 in the form of cylinders, and the monitoring and control of pressure and/or fluid flow through inflatable members 204.

FIG. 2B illustrates a urinary control device 200b having an electronic pump assembly 206b according to an aspect. In some examples, the urinary control device 200b is an artificial urinary sphincter device. The electronic pump assembly 206b may include any of the features of the electronic pump assembly discussed herein, including fluid control system 206 of FIG. 2A. The example urinary control device 200b may be an example of the medical device 100 of FIGS. 1A to IF and may include any of the details discussed with reference to those figures. The urinary control device 200b includes a pump assembly 206b, a fluid reservoir 202b, and a cuff 204b (e.g., an inflatable cuff). In some examples, the urinary control device 200b may be charged using the power transmission device 150 of FIGS. 1A to IF. In some examples, the power receiver 130 (including the receiver member 132), the electronic circuitry 134, battery 136, and/or the sensor(s) 141 are included in the pump assembly 206b.

The fluid reservoir 202b may be a pressure-regulating inflation balloon or element. The fluid reservoir 202b is in operative fluid communication with the cuff 204b via one or more tube members 203b, 205b. The fluid reservoir 202b is constructed of polymer material that is capable of elastic deformation to reduce fluid volume within the fluid reservoir 202b and push fluid out of the fluid reservoir 202b and into the cuff 204b. However, the material of the fluid reservoir 202b can be biased or include a shape memory construct adapted to generally maintain the fluid reservoir 202b in its expanded state with a relatively constant fluid volume and pressure. In some examples, this constant level of pressure exerted from the fluid reservoir 202b to the cuff 204b will keep the cuff 204b at a desired inflated state when open fluid communication is provided between the fluid reservoir 202b and the cuff 204b. In some examples, the fluid reservoir 202b is implanted into the abdominal space.

A user may use an external device 220b to control the urinary control device 200b. In some examples, the user may use the external device 220b to inflate or deflate the cuff 204b. For example, in response to the user activating an inflation cycle using the external device 220b, the external device 220b may transmit a wireless signal to the electronic pump assembly 206b to initiate the inflation cycle to transfer fluid from the fluid reservoir 202b to the cuff 204b (e.g., by opening an active valve where the pressure in the fluid reservoir 202b causes the fluid to move through the active valve to the cuff 204b). In some examples, in response to the user activating a deflation cycle using the external device 220b, the external device 220b may transmit a wireless signal to the electronic pump assembly 206b to initiate the deflation cycle to transfer fluid from the cuff 204b to the fluid reservoir 202b.

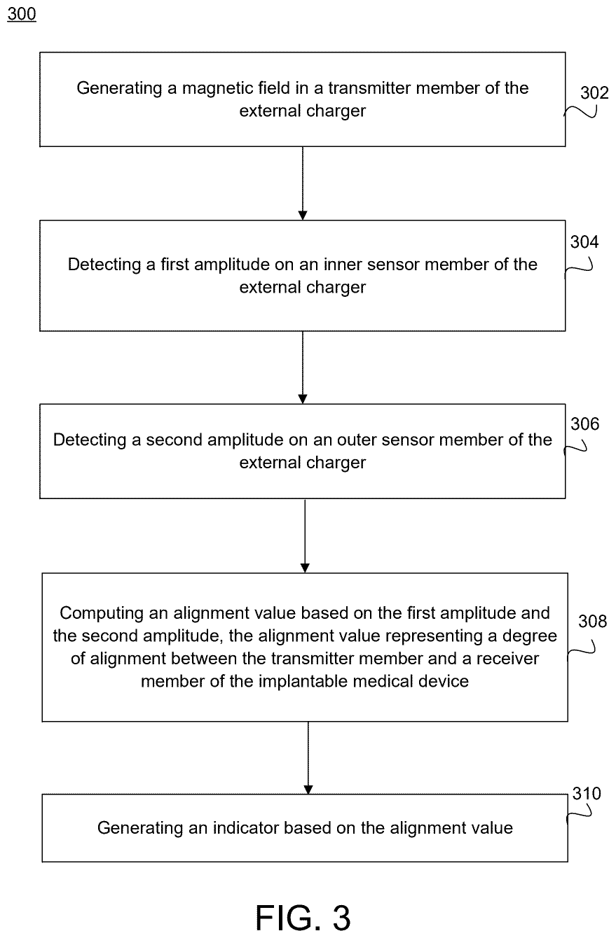

FIG. 3 is a flowchart of an example process 300 for aligning a power transmission device with an implantable medical device to wirelessly charge the implantable medical device.

Operation 302 includes generating a magnetic field in a transmitter member of the external charger. Operation 304 includes detecting a first amplitude on an inner sensor member of the external charger. Operation 306 includes detecting a second amplitude on an outer sensor member of the external charger. Operation 308 includes computing an alignment value based on the first amplitude and the second amplitude, the alignment value representing a degree of alignment between the transmitter member and a receiver member of the implantable medical device. Operation 310 includes generating an indicator based on the alignment value.

Clause 1. An external charger for an implantable medical device, the external charger comprising: a power converter configured to generate a magnetic field on a transmitter member of the external charger; an alignment detector configured to: detect a first amplitude on an inner sensor member of the external charger; detect a second amplitude on an outer sensor member of the external charger; and compute an alignment value based on the first amplitude and the second amplitude, the alignment value representing a degree of alignment between the transmitter member and a receiver member of the implantable medical device; and an indicator generator configured to generate an indicator based on the alignment value.

Clause 2. The external charger of clause 1, wherein the alignment detector includes a peak detector circuit configured to generate a first voltage waveform from a sense coil voltage on the inner sensor member and generate a second voltage waveform from a sense coil voltage on the outer sensor member.

Clause 3. The external charger of clause 2, wherein the alignment detector includes a controller configured to detect the first amplitude from the first voltage waveform and detect the second amplitude from the second voltage waveform.

Clause 4. The external charger of clause 3, wherein the controller is configured to sample the first voltage waveform and the second voltage waveform to detect the first amplitude and the second amplitude, respectively.

Clause 5. The external charger of any one of clauses 1 to 4, wherein the alignment detector includes a voltage divider connected to the inner sensor member and the outer sensor member.

Clause 6. The external charger of any of clauses 1 to 5, wherein the power converter includes a first switch and a second switch, the external charger further comprising: a pulse width modulator configured to generate a control signal; and activating the first switch and the second switch based on the control signal.

Clause 7. An apparatus comprising: an implantable medical device including a receiver member; and an external charger having a transmitter member, an inner sensor member, and an outer sensor member, the external charger configured to: generate a magnetic field on the transmitter member; detect a first amplitude on the inner sensor member; detect a second amplitude on the outer sensor member; and compute an alignment value based on the first amplitude and the second amplitude, the alignment value representing a degree of alignment between the transmitter member and the receiver member; and generate an indicator based on the alignment value.

Clause 8. The apparatus of clause 7, wherein the external charger is configured to compute the alignment value as a ratio of the first amplitude and the second amplitude.

Clause 9. The apparatus of clause 7 or 8, wherein the implantable medical device includes a urology medical implant.

Clause 10. The apparatus of any one of clauses 7 to 9, wherein the external charger is configured to a strength of the magnetic field while charging the implantable medical device.

Clause 11. A method for aligning an external charger and an implantable medical device, the method comprising: generating a magnetic field in a transmitter member of the external charger; detecting a first amplitude on an inner sensor member of the external charger; detecting a second amplitude on an outer sensor member of the external charger; computing an alignment value based on the first amplitude and the second amplitude, the alignment value representing a degree of alignment between the transmitter member and a receiver member of the implantable medical device; and generating an indicator based on the alignment value.

Clause 12. The method of clause 11, wherein computing the alignment value includes computing a ratio of the first amplitude and the second amplitude.

Clause 13. The method of clause 11 or 12, further comprising: generating, by a peak detector circuit, a voltage waveform from a sense coil voltage on the inner sensor member; and detecting, by a controller, the first amplitude from the voltage waveform.

Clause 14. The method of clause 13, further comprising: sampling, by the controller, the voltage waveform to detect the first amplitude.

Clause 15. The method of clause 13, further comprising: detecting the sense coil voltage using a voltage divider connected to the inner sensor member.

Clause 16. A method for aligning an external charger and an implantable medical device, the method comprising: generating a magnetic field in a transmitter member of the external charger; detecting a first amplitude on an inner sensor member of the external charger; detecting a second amplitude on an outer sensor member of the external charger; computing an alignment value based on the first amplitude and the second amplitude, the alignment value representing a degree of alignment between the transmitter member and a receiver member of the implantable medical device; and generating an indicator based on the alignment value.

Clause 17. The method of clause 16, wherein computing the alignment value includes computing a ratio of the first amplitude and the second amplitude.

Clause 18. The method of clause 16, further comprising: generating, by a peak detector circuit, a voltage waveform from a sense coil voltage on the inner sensor member; and detecting, by a controller, the first amplitude from the voltage waveform.

Clause 19. The method of clause 18, further comprising: sampling, by the controller, the voltage waveform to detect the first amplitude.

Clause 20. The method of clause 18, further comprising: detecting the sense coil voltage using a voltage divider connected to the inner sensor member.

Clause 21. The method of clause 16, further comprising: generating, by a peak detector circuit, a voltage waveform from a sense coil voltage on the outer sensor member; and detecting, by a controller, the second amplitude from the voltage waveform.

Clause 22. The method of clause 21, further comprising: sampling, by the controller, the voltage waveform to detect the second amplitude.

Clause 23. The method of clause 21, further comprising: detecting the sense coil voltage using a voltage divider connected to the outer sensor member.

Clause 24. The method of clause 16, further comprising: adjusting a strength of the magnetic field.

Clause 25. The method of clause 24, further comprising: detecting a temperature of the transmitter member; and adjusting the strength of the magnetic field based on the temperature.

Clause 26. An external charger for an implantable medical device, the external charger comprising: a power converter configured to generate a magnetic field on a transmitter member of the external charger; an alignment detector configured to: detect a first amplitude on an inner sensor member of the external charger; detect a second amplitude on an outer sensor member of the external charger; and compute an alignment value based on the first amplitude and the second amplitude, the alignment value representing a degree of alignment between the transmitter member and a receiver member of the implantable medical device; and an indicator generator configured to generate an indicator based on the alignment value.

Clause 27. The external charger of clause 26, wherein the alignment detector includes a peak detector circuit configured to generate a first voltage waveform from a sense coil voltage on the inner sensor member and generate a second voltage waveform from a sense coil voltage on the outer sensor member.

Clause 28. The external charger of clause 27, wherein the alignment detector includes a controller configured to detect the first amplitude from the first voltage waveform and detect the second amplitude from the second voltage waveform.

Clause 29. The external charger of clause 28, wherein the controller is configured to sample the first voltage waveform and the second voltage waveform to detect the first amplitude and the second amplitude, respectively.

Clause 30. The external charger of clause 26, wherein the alignment detector includes a voltage divider connected to the inner sensor member and the outer sensor member.

Clause 31. The external charger of clause 26, wherein the power converter includes a first switch and a second switch, the external charger further comprising: a pulse width modulator configured to generate a control signal; and activating the first switch and the second switch based on the control signal.

Clause 32. An apparatus comprising: an implantable medical device including a receiver member; and an external charger having a transmitter member, an inner sensor member, and an outer sensor member, the external charger configured to: generate a magnetic field on the transmitter member; detect a first amplitude on the inner sensor member; detect a second amplitude on the outer sensor member; and compute an alignment value based on the first amplitude and the second amplitude, the alignment value representing a degree of alignment between the transmitter member and the receiver member; and generate an indicator based on the alignment value.

Clause 33. The apparatus of clause 32, wherein the external charger is configured to compute the alignment value as a ratio of the first amplitude and the second amplitude.

Clause 34. The apparatus of clause 32, wherein the implantable medical device includes a urology medical implant.

Clause 35. The apparatus of clause 32, wherein the external charger is configured to a strength of the magnetic field while charging the implantable medical device.

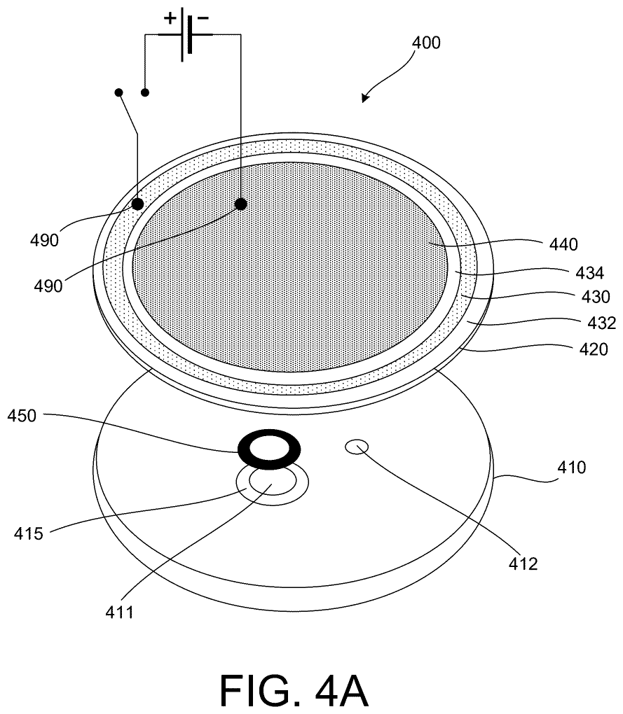

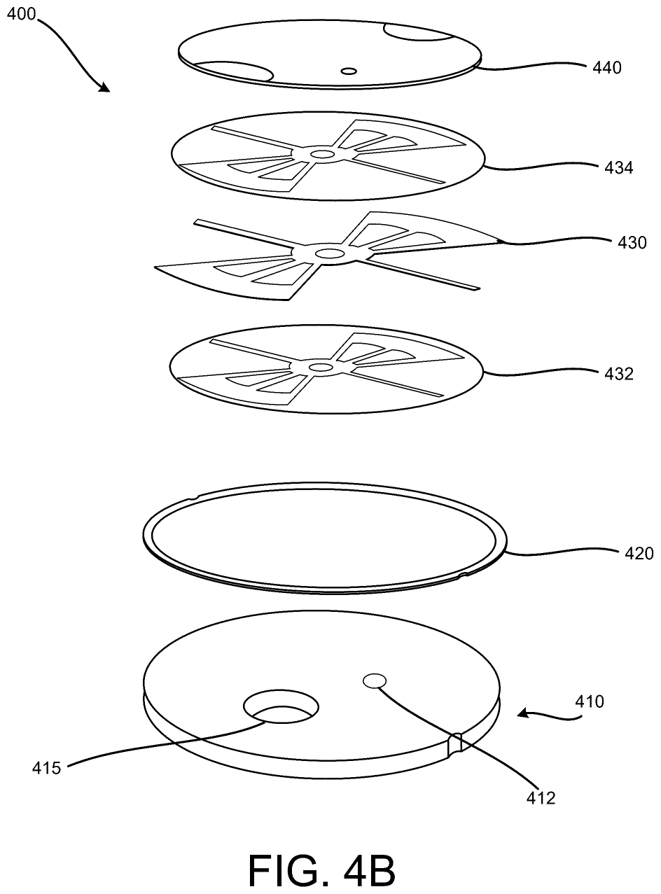

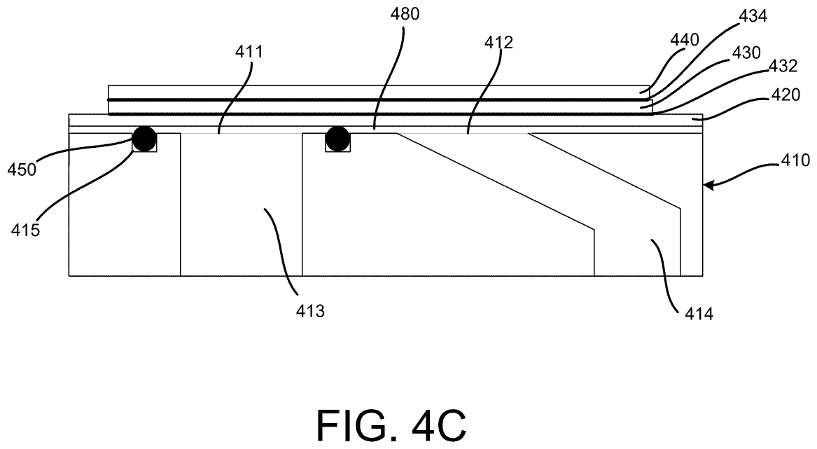

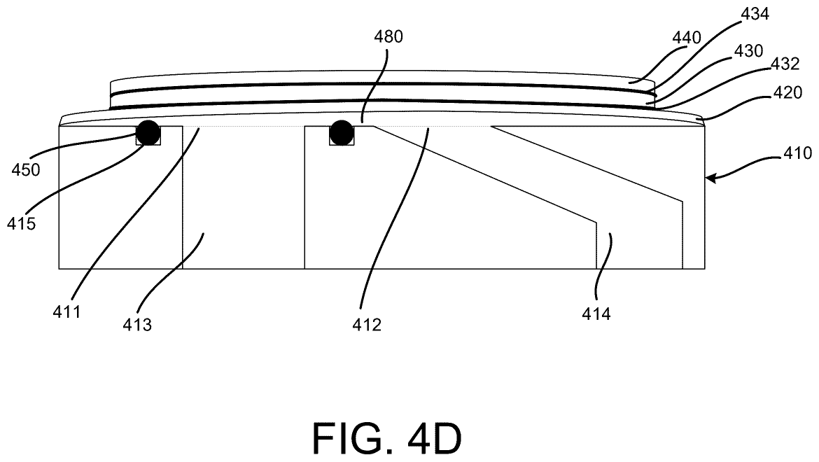

FIG. 4A is a partially exploded perspective view of an example valve device 400. FIG. 4B is an exploded perspective view of the example valve device 400. FIGS. 4C and 4D are cross-sectional views of the example valve device 400 shown in FIG. 4A, in an assembled state. The example valve device 400 shown in FIGS. 4A-4D may be an example of the medical device 100 of FIGS. 1A to IF and may include any of the details discussed herein.

In the example arrangement shown in FIGS. 4A-4D, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. The piezoelectric element can be electrically powered (e.g., by a battery in the implantable fluid-operated inflatable device 100) to drive the diaphragm 420 to open and close the valve device 400. The diaphragm 420 can include a thin metal foil, whose shape can be repeatedly deformed in response to movement by the piezoelectric element 440. In some implementations, the diaphragm 420 can include titanium material. In some implementations, the diaphragm 420 can include gold material. In some implementations, the diaphragm 420 can include stainless steel material or other alloys. In some implementations, the isolation layer 430 can include a polyamide material that has a high resistivity, for example, a resistivity greater than 1013 Ohm-cm to provide electrical isolation between the piezoelectric element 440 and the diaphragm 420.

In some examples, an epoxy layer 432 provides for the coupling of the isolation layer 430 and the diaphragm 420. In some examples, an epoxy layer 434 provides for the coupling of the piezoelectric element 440 and the isolation layer 430, and the epoxy layers 432, 434 together provide for the coupling of the piezoelectric element 440 to the diaphragm 420. In some implementations, the epoxy layers 432, 434 are not distinct but are part of one epoxy layer. The epoxy layers 432, 434 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of a voltage to the piezoelectric element 440 causes a deflection or deformation of the piezoelectric element 440 and a corresponding deflection or deformation of the diaphragm 420 coupled thereto.

In the example arrangement shown in FIGS. 4A-4D, a fluid chamber 480 is defined between the base plate 410 and the diaphragm 420. For example, in some implementations, the diaphragm 420 can be bonded to the base plate 410 at the periphery of the diaphragm to form a fluid-tight connection between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4D, the base plate 410 includes a recess 415 surrounding the first opening 411, with a seal 450, in the form of an O-ring in the example shown in FIGS. 4A-4D, fitted in the recess 415. In some examples, a top portion of the seal 450 is pressed against the diaphragm 420 in the closed position of the valve device 400, as shown in FIG. 4C to close off the chamber 480 and inhibit the flow of fluid through the example valve device 400, between the first fluid passageway 413 and the second fluid passageway 414 via the chamber 480. In some examples, in which the valve device 400 does not include a seal 450, the diaphragm 420 is seated against the base plate 410 to close off the chamber 480 and inhibit the flow of fluid through the valve device 400. In the open position of the example valve device 400, the base plate 410 and the top portion of the seal 450 are separated, or spaced apart from, the diaphragm 420 due to the deflection of the diaphragm 420. This positioning of the seal 450 and the base plate 410 relative to the diaphragm 420 opens the chamber 480 and allows fluid to flow through the example valve device 400, between the first fluid passageway 413 and the second fluid passageway 414 via the fluid chamber 480.

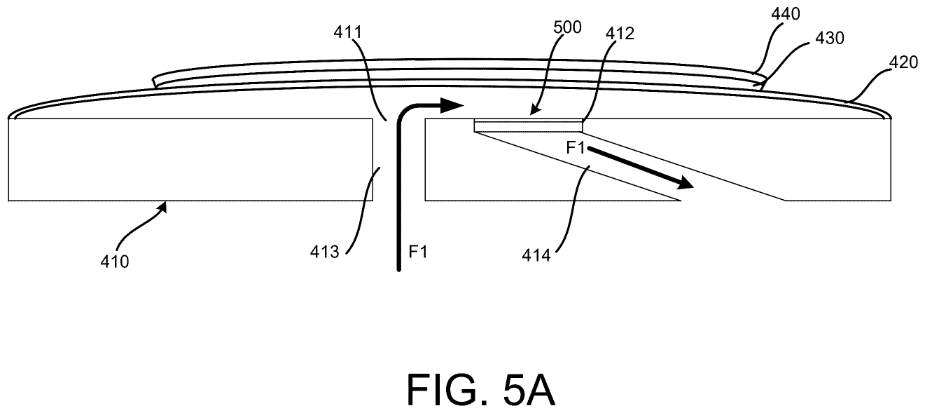

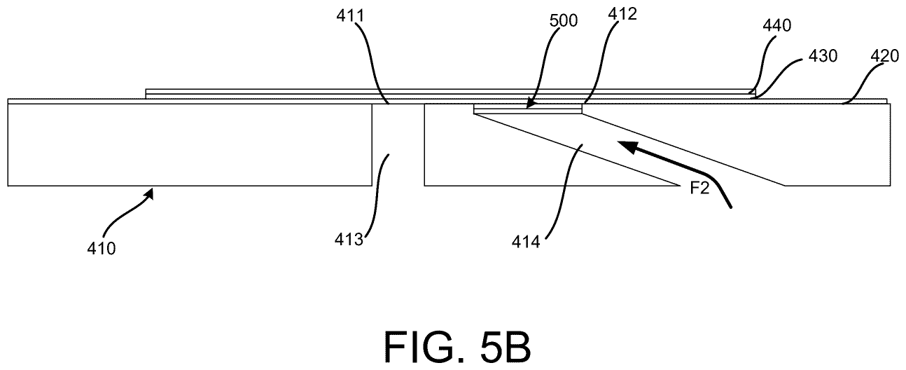

FIGS. 5A and 5B are cross-sectional views of the example valve device 400 shown in FIGS. 4A-4D, including an example flow control device 500 positioned in one of the fluid passageways of the example valve device 400.

FIG. 5A illustrates an example in which the valve device 400 is open, allowing fluid to flow in the direction of the arrows F1, through the first fluid passageway 413, into the chamber 480, and out of the valve device 400 through the second fluid passageway 414. The example shown in FIG. 5A may illustrate an open position of the valve device 400 that allows fluid to flow, for example, from the reservoir 202 to the inflatable member 204 to provide for inflation/pressurization of the inflatable member 204.

In the example arrangement shown in FIGS. 5A and 5B, the example flow control device 500 is positioned at the second opening 412 formed in the base plate 410, the second opening 412 providing for fluid communication between the fluid chamber 480 and the second fluid passageway 414. In some examples, the flow control device 500 is a check valve, or a one-way valve, that allows for flow in one direction (in this example, in the direction of the arrows F1), while inhibiting flow in the opposite direction.

FIG. 5B illustrates the closed position of the valve device 400, in which the flow of fluid through the valve device 400 is blocked. In some examples, the closed position shown in FIG. 5B may maintain an inflation pressure of the inflatable member 204. As described above, in some situations, pressure fluctuations and/or pressure spikes may exert a force, or pressure on the valve device 400 in the closed position. FIG. 5B illustrates a pressure spike, or a back pressure, exerted in the direction of the arrow F2. In the example described above with respect to FIGS. 4A-4D, this type of pressure spike, or back pressure exerted on the diaphragm 420/piezoelectric element 440 could cause an unintentional opening of the valve device 400, and an unintentional deflation/depressurization of the inflatable member 204. In the example shown in FIG. 5B, the flow control device 500 (positioned at the second opening 412, between the second fluid passageway 414 and the fluid chamber 480), for example, in the form of a check valve or a one-way valve, remains in the closed position in response to the pressure spike/back pressure/flow of fluid in the direction of the arrow F2. Thus, the positioning of the flow control device 500 at the second opening 412, allowing flow in a first direction, i.e., the direction of the arrows F1, while blocking flow in a second direction, i.e., the direction of the arrow F2, maintains the closed state of the valve device 400, even in response to fluctuation in pressure, or pressure spike, or back pressure.

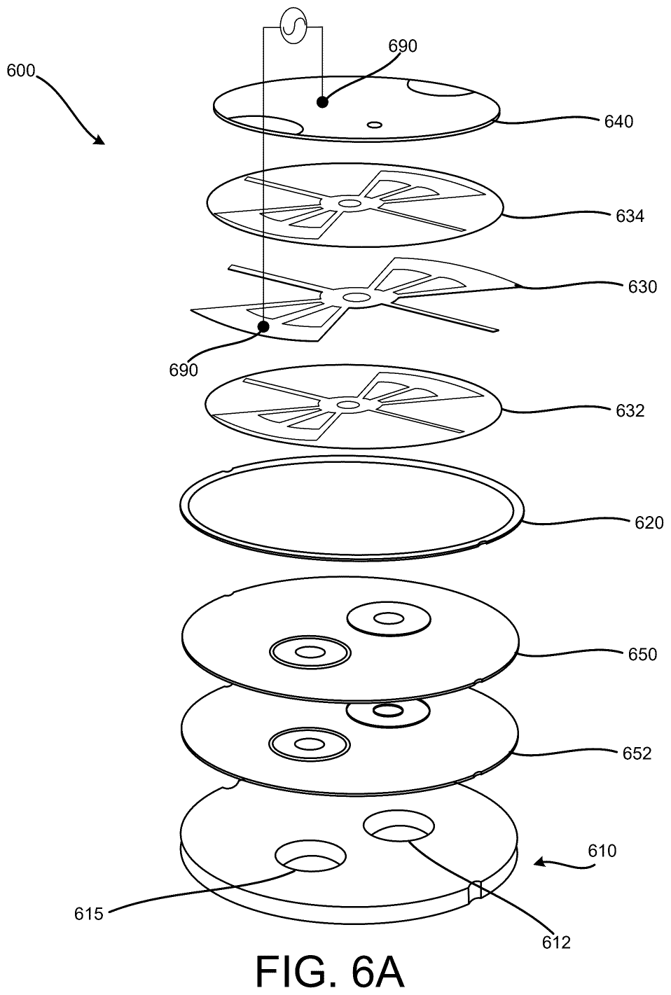

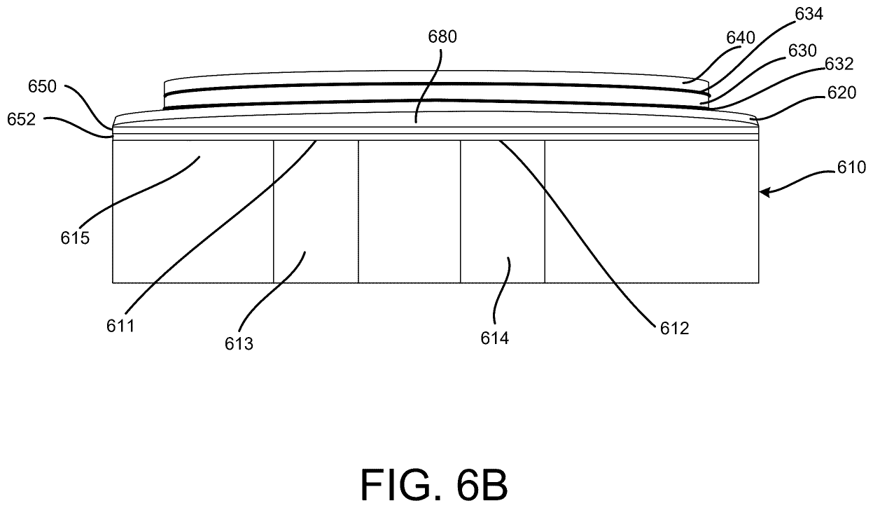

FIG. 6A is a partially exploded perspective view of an example pump device 600, and FIG. 6B is a cross-sectional view of the example pump device 600. The example pump device 600 shown in FIGS. 6A-6B is an example of a fluid control device, or a fluidic component, included in the fluid control system 206 of the example electronically controlled fluid manifold 230 described above.

In the example arrangement shown in FIGS. 6A-6B, the example pump device 600 includes a base plate 610 defining a base portion of the pump device 600. A diaphragm 620 is positioned on the base plate 610. A piezoelectric element 640 is positioned on the diaphragm 620, with an isolation layer 630 positioned between the diaphragm 620 and the piezoelectric element 640. The piezoelectric element can be electrically powered (e.g., by a battery (e.g., the battery 136 of FIG. 1A) of the implantable fluid-operated inflatable device 100) to drive the diaphragm 620 to pump fluid through the pump device 600. The diaphragm 620 can include a thin metal foil, whose shape can be repeatedly deformed in response to movement by the piezoelectric element 640. In some implementations, the diaphragm 620 can include titanium material. In some implementations, the diaphragm 620 can include gold material. In some implementations, the diaphragm 620 can include stainless steel material or other alloys. In some implementations, the isolation layer 630 can include a polyamide material that has a high resistivity, for example, a resistivity greater than 1013 Ohm-cm to provide electrical isolation between the piezoelectric element 640 and the diaphragm 620.

In some examples, an epoxy layer 632 provides for the coupling of the isolation layer 630 and the diaphragm 620. In some examples, an epoxy layer 634 provides for the coupling of the piezoelectric element 640 and the isolation layer 630, and the epoxy layers 632, 634 together provide for the coupling of the piezoelectric element 640 to the diaphragm 620. In some implementations, the epoxy layers 632, 634 are not distinct but are part of one epoxy layer. The epoxy layers 632, 634 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 690 are arranged on the example pump device 600. In the example shown in FIG. 6A, the example pump device 600 includes a pair of electrodes 690 coupled between the isolation layer 630 and the piezoelectric element 640. Application of a voltage to the piezoelectric element 640 causes a deflection or deformation of the piezoelectric element 640 and a corresponding deflection or deformation of the diaphragm 620 coupled thereto.

When the pump device 600 is used in the fluid control system 206 of the example electronically controlled fluid manifold 230 described above, the piezoelectric element 640 can be controlled to cause fluid to be pumped by pump device 600, for example, by repeatedly changing a volume of the fluid chamber 680 by deforming the deformable diaphragm 620 to pump fluid from the fluid reservoir to the inflatable member.

In the example arrangement shown in FIGS. 6A-6B, a fluid chamber 680 is defined between the base plate 610 and the diaphragm 620. The base plate 610 includes a first opening 615 that provides for communication between a first fluid passageway 613 and the fluid chamber 680. The base plate 610 includes a second opening 612 that provides for communication between a second fluid passageway 614 and the fluid chamber 680. In some examples, the diaphragm 620 can be actuated to move between a closed position in which the diaphragm 620 is proximate to the base plate 610 due to the deflection of the diaphragm 620, such that the volume of the chamber 680 is minimized, and an open position in which the base plate 610 is separated, or spaced apart from, the diaphragm 620 due to the deflection of the diaphragm 620, such that the volume of the chamber is maximized. When the diaphragm 620 is actuated to move from the closed position to the open position, fluid can be drawn into the chamber 680 through the first fluid passageway 613, and when the diaphragm 620 is actuated to move from the open position to the closed position, fluid can be expelled from the chamber 680 through the second fluid passageway 614. Repeatedly actuating the diaphragm between the closed and open position allows fluid to be pumped through the pump device 600, from the first fluid passageway 613 to the second fluid passageway 614 via the fluid chamber 680.

In some implementations, the pump device 600 can include one or more foil plates 650 and 652 to control the flow of fluid into and out of the pump device 600. The foil plates 650, 652 can include one-way check valves that operate to permit fluid to flow in one direction through the values but not in an opposite direction. The one-way check valves defined by the one or more foil plates can be positioned in, or in fluid connection with, a fluid passageway 613, 614 of the pump device 600. In some examples, a check valve is positioned in, or in fluid connection with, a portion of a fluid passageway 613, 614 so as to inhibit the unintended flow of fluid through the pump device in the event of a fluctuation, or spike in pressure. In some examples, a check valve is positioned in a fluid passageway 613, 614 so as to counteract a back pressure that would otherwise overcome the closing pressure and cause unintentional flow through the pump device 600. In some example implementations, a first check valve defined by one or more foil plates 650, 652 is positioned in, or in fluid connection with (e.g., at a first opening 611 of), a first fluid passageway 613 of the pump device and is configured to permit fluid to easily flow from the first fluid passageway 613 into the chamber 680 but to prevent or inhibit the flow of fluid from the chamber 680 into the first fluid passageway 613. In some example implementations, a second check valve defined by one or more foil plates 650, 652 is positioned in, or in fluid connection with (e.g., at a first opening 615 of), a second fluid passageway 614 of the pump device 600 and is configured to permit fluid to easily flow from the chamber 680 into the second fluid passageway 614 but to prevent or inhibit the flow of fluid from the first fluid passageway 613 into the chamber 680.

Application of an alternating current (AC) voltage to the piezoelectric element 640 can cause the diaphragm 620 of the pump device 600 to oscillate between a first position that defines the closed position of the chamber 680, in which the diaphragm 620 is proximate to the base plate 610 and the volume of the chamber 680 is minimized, and a second (e.g., domed) position that defines the open position of the chamber 680, in which the diaphragm 620 is separated from the base plate and the volume of the chamber 680 is maximized. As the diaphragm 620 of the pump device 600 oscillates between a first position and the second position, fluid is drawn into the chamber 680 from the first passageway 613 and is expelled from the chamber 680 into the second passageway 614. As the diaphragm 620 of the pump device 600 oscillates between a first position and the second position, the one-way check valves defined by the one or more foil plates 650, 652 prevent or inhibit fluid from flowing from the chamber 680 into the first passageway 613 and prevent or inhibit fluid from flowing into the chamber 680 from the second passageway 614. Thus, the application of the AC voltage to the piezoelectric element 640 causes the pump device 600 to pump fluid from the first passageway 613 to the second passageway 614.

The frequency of the AC voltage applied to the piezoelectric element 640 can determine an oscillation mode of the piezoelectric element 640. In some implementations, the frequency of the AC voltage is selected to excite a lowest-order mode in which the center of the circular piezoelectric element 640 experiences the greatest extent of movement during an oscillation cycle, such that an amount of fluid pumped during an oscillation cycle is maximized compared to other oscillation modes.

The piezoelectric element 640 can be controlled to cause fluid to be pumped by device 600, for example, by repeatedly changing a volume of the fluid chamber 680 by deforming the deformable diaphragm 620 to pump fluid from the fluid reservoir to the inflatable member.

The volume of the chamber 680 can be determined, at least in part, by the shape, geometry, and material properties of the components used to form the chamber 680, including, for example, the base plate 610 and the deformable diaphragm 620. In some cases, a relatively larger volume of the chamber 680, for an approximately constant diameter of the chamber, can result in more fluid being pumped in each open/close cycle of the pump 600. To achieve a relatively larger volume of chamber 680, the deformable diaphragm can be deformed or biased into a non-flat dome-shaped configuration before it is attached to the piezoelectric element 640.

In some implementations, before the diaphragm 620 is placed in attached to the piezoelectric element 640, a voltage can be placed across the electrodes 690 attached to the piezoelectric element 640 to configure the piezoelectric element 640 in the domed configuration that is assumed when the fluid chamber is in the open position (See FIG. 4D). Then, the diaphragm can be placed in contact with the piezoelectric element while the piezoelectric element 440 is in its domed configuration, and the epoxy can be cured when the piezoelectric element and the diaphragm 420 are in the domed configuration, which can reduce stress on the adhesive bond between the diaphragm 420 and the piezoelectric element 440.

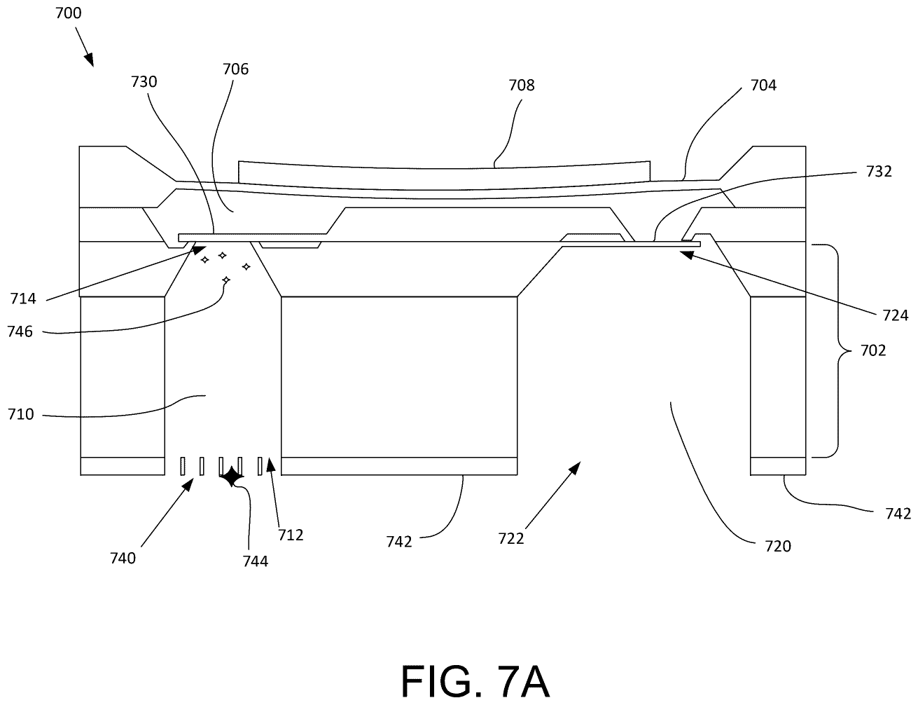

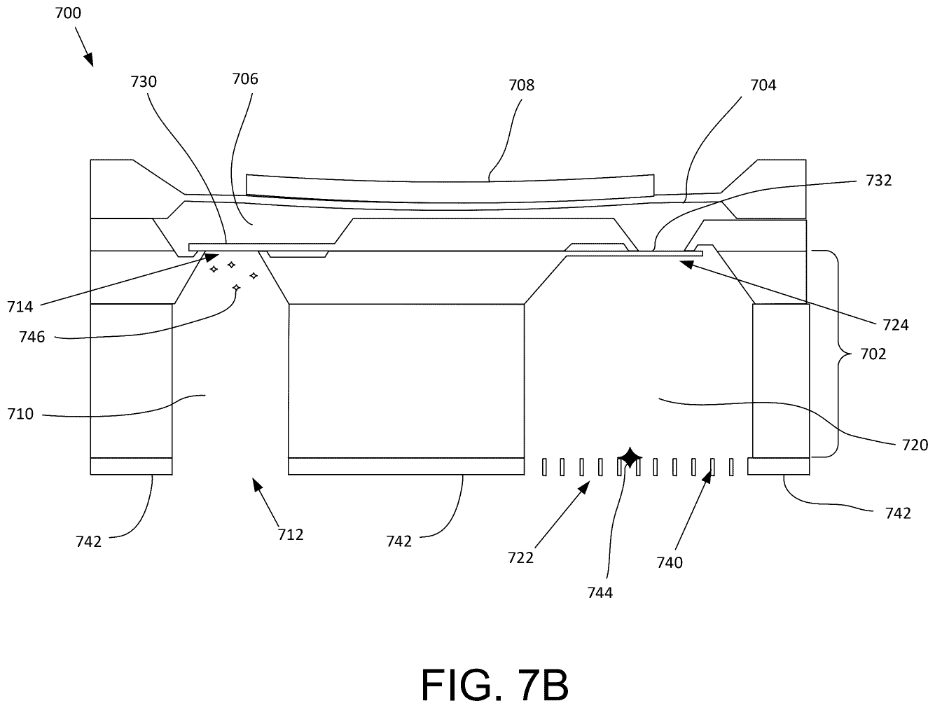

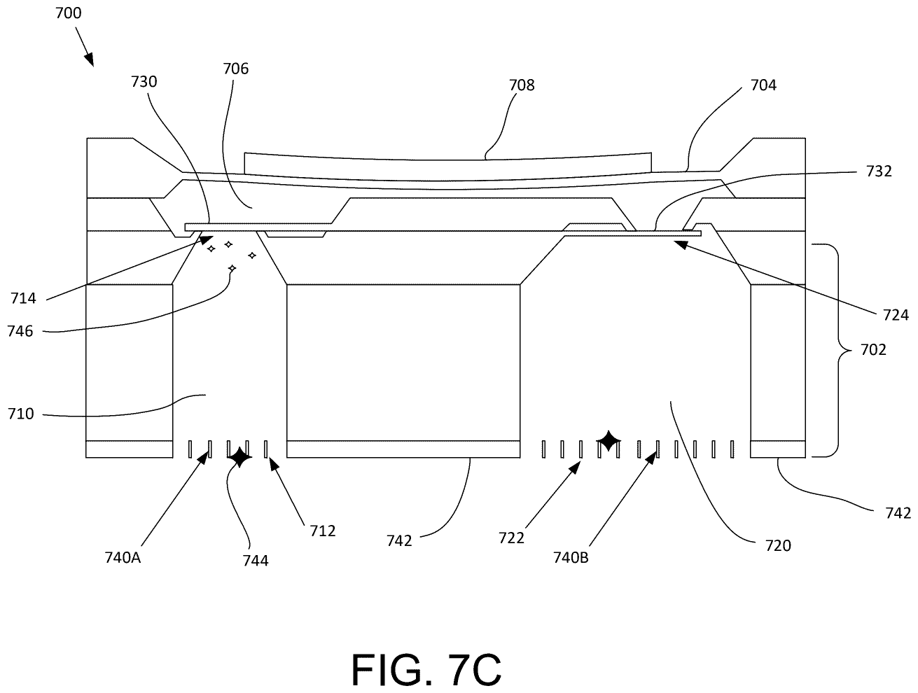

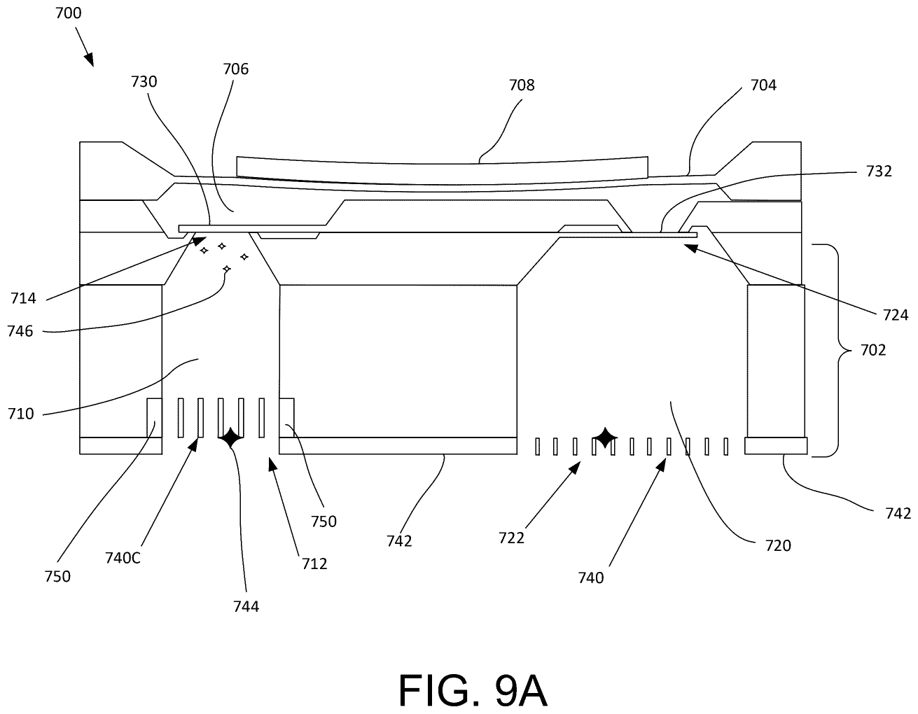

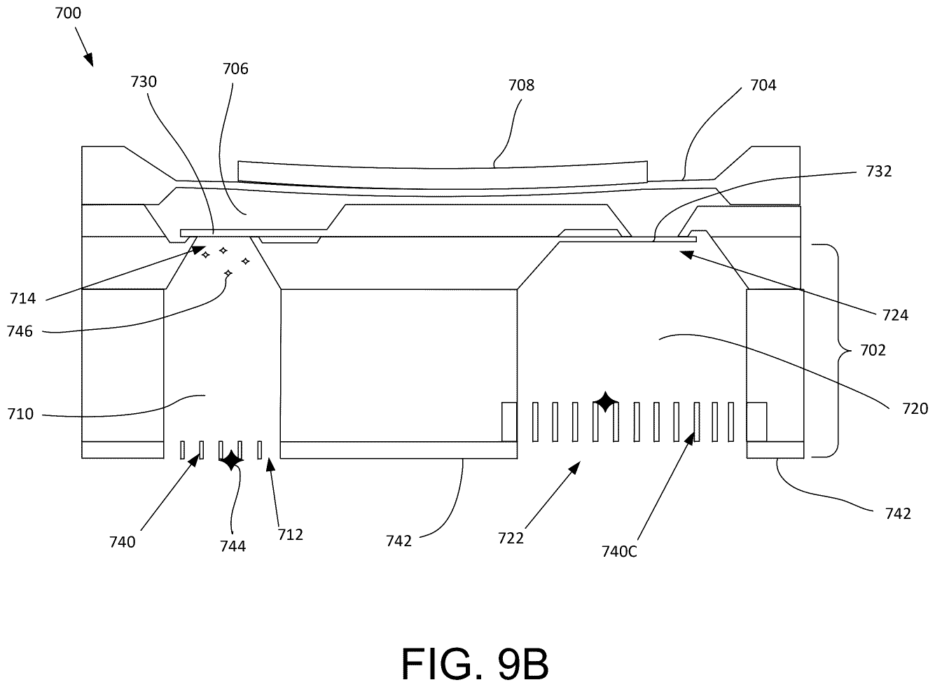

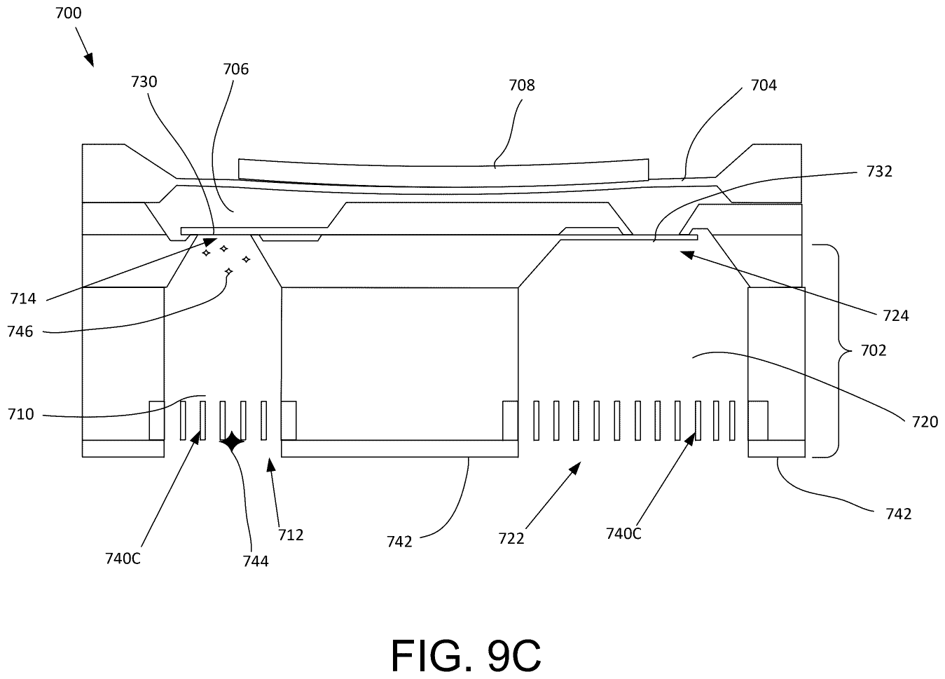

FIGS. 7A, 7B, 7C, 9A, 9B, and 9C are cross-sectional views of example pump devices 700 that includes a filter for capturing particulate matter in the fluid flow and/or for blocking the particulate matter from entering certain parts of the fluidic system (e.g., for blocking particulate matter from entering a pump chamber of the device). The example pump device 700 shown in FIGS. 7A, 7B, 7C, 9A, 9B, and 9C are examples of a fluid control device, or a fluidic component, included in the fluid control system 206 of the example electronically controlled fluid manifold 230 described above.

In the example arrangements shown in FIGS. 7A, 7B, 7C, 9A, 9B, and 9C, the example pump device 700 includes a base plate 702 defining a base portion of the pump device 700. A diaphragm 704 is positioned above the base plate 702, and a fluid chamber 706 is defined between the base plate 702 and the diaphragm 704. A piezoelectric element 708 is positioned on the diaphragm 704. The piezoelectric element can be electrically powered (e.g., by a battery of the implantable fluid-operated inflatable device) to drive the diaphragm 704 to pump fluid through the pump device 700. The diaphragm 704 can include a thin metal foil, whose shape can be repeatedly deformed in response to movement by the piezoelectric element 708. In some implementations, the diaphragm 704 can include titanium material.

The base plate 702 can define a first fluid passageway 710 through which fluid can flow from a fluid reservoir into the fluid chamber 706. The first fluid passageway 710 can include an opening 712 at a first end of the passageway 710, which is distal to the fluid chamber 706, and can include an opening 714 and a second end of the passageway 710, which is proximate to the fluid chamber 706. The base plate 702 can define a second fluid passageway 720 through which fluid can flow from the fluid chamber 706 to an inflatable member. The second fluid passageway 720 can include an opening 722 at a first end of the second fluid passageway 720, which is distal to the fluid chamber 706, and can include an opening 724 and a second end of the second fluid passageway 720, which is proximate to the fluid chamber 706. In some implementations, the first fluid passageway 710 and the second fluid passageway 720 can be tapered, such the passageways 710, 720 have larger cross-sectional areas at the ends of the passageways that are distal to the fluid chamber 706 than at ends of the passageways that are proximate to the fluid chamber.

The pump device 700 can include a first flexible flap 730 that includes a portion that has an area that is greater than an area of the passageway opening 714 that is proximate to the fluid chamber 706 and that covers the opening, such that the first flexible flap 730 is configured to seal against portions of the base plate that defines the opening 714 of the first fluid passageway 710 to close the opening 714 when a fluid pressure in the fluid chamber 706 is greater than a fluid pressure of fluid in the first fluid passageway 710. The flexible flap 730 can be secured to the base plate over a portion of its extent but can have a portion that is unsecured, such that at least a portion of the flexible flap is configured to be pushed away from one or more walls of the fluid passageway 710 that defines the opening 714 when a fluid pressure of fluid in the first fluid passageway 710 is greater than a fluid pressure in the fluid chamber 706. In this manner, the flexible flap 730 operates to allow fluid to flow from the first fluid passageway 710 into the fluid chamber 706 but to block the flow of fluid from the fluid chamber 706 into the first fluid passageway 710. The flexible flap 730 can be made of a variety of materials including, for example, titanium, elastomeric material, plastic material, etc.

The pump device 700 can include a second flexible flap 732 that includes a portion that has an area that is greater than an area of the passageway opening 724 that is proximate to the fluid chamber 706 and that covers the opening, such that the second flexible flap 732 is configured to seal against portions of the base plate that defines the opening 724 of the second fluid passageway 720 to close the opening 724 when a fluid pressure in the fluid chamber 706 is greater than a fluid pressure of fluid in the second fluid passageway 720. The flexible flap 732 can be secured to the base plate over a portion of its extent but can have a portion that is unsecured, such that at least a portion of the flexible flap is configured to be pushed away from one or more walls of the second fluid passageway 720 that defines the opening 724 when a fluid pressure in the fluid chamber 706 is greater than a fluid pressure of fluid in the second fluid passageway 720. In this manner, the flexible flap 732 operates to allow fluid to flow from the fluid chamber 706 into the second fluid passageway 720 but to block the flow of fluid from the second fluid passageway 720 into the fluid chamber 706. The flexible flap 732 can be made of a variety of materials including, for example, titanium, elastomeric material, plastic material, etc.

With the flexible flaps 730, 732 configured in this way to allow fluid to flow in a first direction from the first fluid passageway 710 into the fluid chamber 706 and out of the fluid chamber into the second fluid passageway 720 but not in a direction opposite to the first direction, repeated expansion and contraction of the volume of the fluid chamber 706 in response to the piezoelectric element 708 operating on the deformable diaphragm 704 can cause fluid to be pumped from a reservoir fluidically connected to the first fluid passageway 710 to an inflatable member that is fluidically connected to the second fluid passageway 720.

The pump device 700 can include a fluid filter 740 that is located within, or at the end of, the first fluid passageway 710 or that is located within, or at the end of, the second fluid passageway 720. The fluid filter 740 can operate to block, for example, debris, foreign matter, particulates suspended in the fluid flowing through the device 700 from passing through the first fluid passageway 710 and into the fluid chamber 706 and/or from exiting the second fluid passageway 720. For example, as shown in FIG. 7A, a fluid filter 740 is located at the opening 712 into the first fluid passageway 710. As shown in FIG. 7B, a fluid filter 740 is located at the opening 722 into the second fluid passageway 720. As shown in FIG. 7C, a fluid filter 740A is located at the opening 712 into the first fluid passageway 710, and a fluid filter 740B is located at the opening 722 into the second fluid passageway 720.

In some implementations, the fluid filter 740, 740A, 740B can include a metal foil (e.g., a titanium foil, having a pattern of openings that permit fluid to flow through the openings but that block particulates having a characteristic size larger than a threshold size from flowing through the opening. For example, particulates 744 having a characteristic size (e.g., minimum transverse extent) that is greater than a threshold size defined by the size (e.g., diameter) of the openings can be blocked by the filter 740, while particulates 746 and a characteristic size smaller than the threshold size can pass through the filter 740.

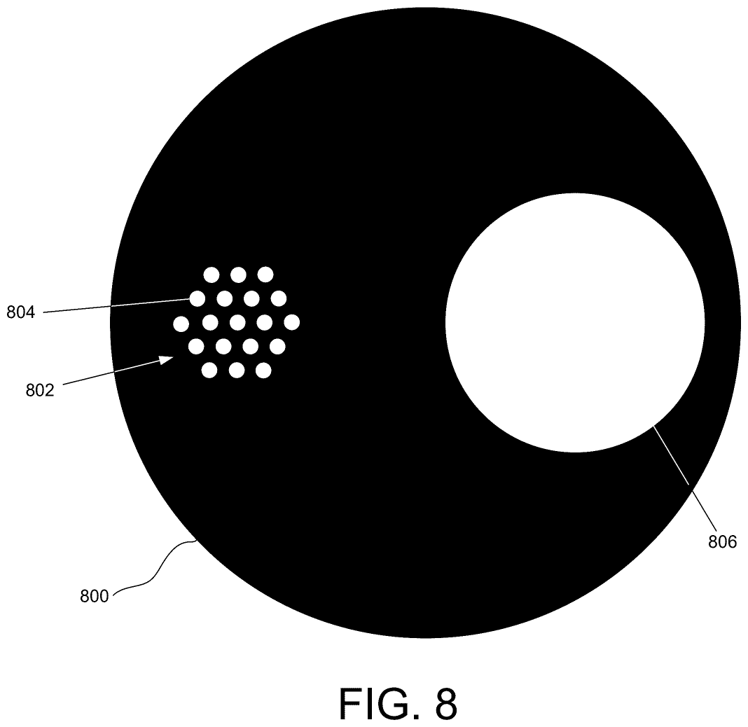

FIG. 8 is a schematic end view of a filter foil 800. In some implementations, the filter foil 800 can be made of metal (e.g., titanium) and can have a first section 802 that includes a plurality of openings 804. The openings can have a variety of different shapes, including circular, oblong, square, rectangular, hexagonal, etc. The plurality of openings 804 can be arranged in a regular or irregular pattern. For example, the openings 804 can be arranged in a two-dimensional hexagonal pattern, as shown in FIG. 8, or in a square pattern, or another type of regular or irregular pattern.

The plurality of openings 804 can be formed in the filter foil 800 in a number of different ways. For example, in some implementations, the pattern of openings can be mechanically stamped into the metal foil 800. In some implementations, the pattern of openings 804 can be laser etched into the metal foil 800. In some implementations, the pattern of openings can be chemically etched (e.g., through a lithographic process) into the metal foil 800.

Referring again to FIG. 7A and also to FIG. 8, the section 802 that includes the plurality of openings 804 can be arranged on the filter foil 800 so that the pattern of openings 804 is aligned with the opening 712 of the first fluid passageway 710 when the filter foil 800 is attached to the base plate 702. The filter foil 800 also can include an opening 806 in the filter foil that is aligned with the opening 722 of the second fluid passageway 720 of the base plate 702 when the filter foil is attached to the base plate.

In some implementations, the filter foil 800 can be welded to the base plate 702. For example, when the base plate includes titanium and the filter foil 800 includes titanium, the filter foil 800 can be welded to the titanium base plate 702. Prior to attempting (e.g., welding) the filter foil 800 to the base plate 702, the filter foil 800 can be positioned relative to the openings in the base plate, such that the first section 802 of the filter foil, which includes the plurality of openings 804, is positioned at the end of the first fluid passageway 710 and such that the opening 806 in the filter foil 800 is positioned at the end of the second fluid passageway 720. Similarly, when a filter foil is attached to the base plate shown in FIG. 7B, a section of the filter foil having a plurality of openings can be aligned with the end of the second fluid passageway 720, and a larger opening in the filter foil 800 in the aligned with the end of the first fluid passageway 710. Similarly, when a filter foil is attached to the base plate shown in FIG. 7C, a first section having a plurality of openings can be aligned with the end of the second fluid passageway 720 and a second section having a plurality of openings can be aligned with the end of the first fluid passageway 710.

In implementations in which the first fluid passageway 710 and the second fluid passageway 720 are tapered, such the passageways 710, 720 have larger cross-sectional areas at the ends of the passageways that are distal to the fluid chamber 706 than at ends of the passageways that are proximate to the fluid chamber, filters 740, 740A, 740B positioned at the distal ends of the fluid passageways 710, 720 can have cross-sectional areas that are greater than the cross-sectional areas of the openings 714, 724 between the passageways 710, 720 and the fluid chamber 706. Because of this the area of the filter that is active for trapping particulate matter can be larger than the areas of the openings 714, 724 between the passageways 710, 720 and the fluid chamber 706. In some implementations the flow of fluid through the filter 740, 740A, 740B can be reversed to dislodge some of the particulate matter that has been trapped by the filters from the filters.

The example pump devices 700 shown in FIGS. 7A, 7B, 7C include filters 740, 740C for blocking particulate matter in the fluid from entering a pump chamber of the device or for circulating in the fluidic system in which the pump devices operate. The filter 740 shown in FIG. 7A is disposed at the distal end of the first fluid passageway 710, and the filter 740 shown in FIG. 7B is disposed at the distal end of the second fluid passageway 720. These filters 740 can be similar to the filters 740, 740B, 740B shown in FIGS. 7A-15C, in that the filters 740 can include a plurality of openings in a foil, where the size of the openings is selected to block the passage of particles having a characteristic size greater than a threshold size and to allow fluid and particles having a characteristic size less than the threshold size to pass through the openings.

In some implementations, the example pump devices 700 shown in FIGS. 7A, 7B, 7C can include filters 740C disposed within the first fluid passageway 710 or within the second fluid passageway 720, for example, between the first end of the first fluid passageway 710 and the opening 714 at the second end of the first fluid passageway 710 and/or between the first end of the second fluid passageway 720 and the opening 724 at the second end of the second fluid passageway 720. For example, as shown in FIG. 7A, the example pump device 700 can include a filter 740C disposed within the first fluid passageway 710. In another example, as shown in FIG. 7B, the example pump device 700 can include a filter 740C disposed within the second fluid passageway 720. In another example, as shown in FIG. 7C, the example pump device 700 can include a filter 740C disposed within the first fluid passageway 710 and another filter 740C disposed within the second fluid passageway 720.

Referring to FIG. 7A, the filter 740C can include an outer frame 750 that supports material within the frame that includes a plurality of small openings or passages through which fluid can pass but which have a threshold size that blocks particles having a characteristic size greater than the threshold size from passing through the filter 740C.

The outer frame 750 can be secured to the base plate 702 that defines the first fluid passageway 710. In some implementations, the base plate 702 can define a receptacle that receives the outer frame 750. In some implementations, the receptacle can have a lateral extent (e.g., a diameter) that is greater than the lateral extent of the first fluid passageway 710, such that when the outer frame 750 is disposed in the receptacle, an inner wall of the outer frame has a lateral extent that is similar to the lateral extent of the first fluid passageway 710. In some implementations, the outer frame can be press fit into the receptacle. In some implementations the outer frame 750 can be welded to the portion of the base plate 702 that defines the receptacle. In some implementations, after the outer frame 750 of the filter 740C is placed in the receptacle, a foil 742 can be placed over the outer frame 750 and then attached (e.g., welded) to the base plate 702.

In different implementations, the outer frame 750 can be made of different materials. For example, if the outer frame 750 is to be welded to a titanium base plate 702, the outer frame 750 can be made of titanium. In another example, if the outer frame 750 is to be securely press fit into a receptacle, the outer frame 750 can be made of a compliant material, for example, plastic, rubber, etc.

The material of the filter 740C supported by the outer frame 750, which includes a plurality of small openings or passages through fluid passes, can be made of different materials, which need not be identical or similar to the materials of the outer frame 750. For example, the material can include metal (e.g., titanium, gold, etc.). In another example the material can include ceramic material. In another example, the material can include plastic.

In some implementations, the thickness of the material of the filter, which includes the plurality of small openings or passages through which fluid passes, in the direction of the fluid flow through the filter can be greater than three times the mean lateral extent of the openings or passages through which the fluid passes. Thus, the openings or passages of the materials can operate more as tubes through which the fluid passes than as apertures in a thin plane of material. In some implementations, walls of the openings or passages of the material can be textured or treated to promote the adhesion of particulate matter, while also permitting the fluid to pass through the openings or passages. For example, the walls of the openings or passages can have a surface texture or roughness that facilitates the adhesion of particulate matter, and the service of the openings or passages can include a hydrophobic coating to encourage the passage of fluid through the openings or passages.

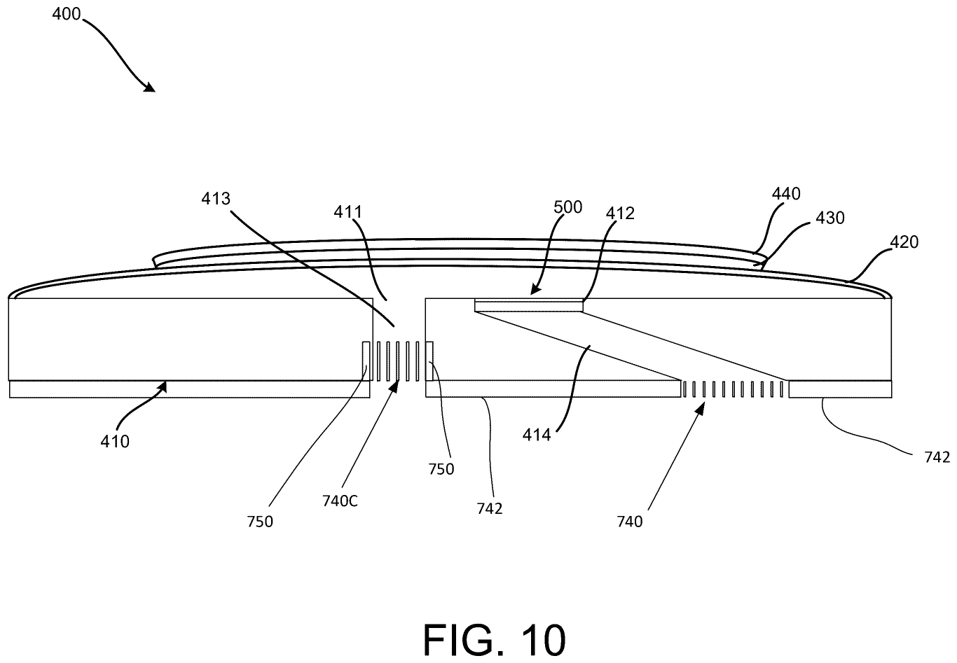

In addition to being used in the pumps described herein, the filters described herein also can be used in the valves described herein. For example, FIG. 10 is cross-sectional view of the valve device 400 shown in FIGS. 5A and 5B, but also including a filter 740 located at an end of the second fluid passageway 414 and a filter 740C located within the first fluid passageway 413. The filters described herein also may be utilized in other valve structures described herein.

It is desirable that the implantable fluid-operated inflatable device described herein can be implanted in a patient and used to provide safe, reliable, and successful therapeutic treatment to the patient for many years, for example, 10 or more years. However, a challenge with meeting this reliability goal is that the piezoelectric elements used in combination with the thin metal diaphragms to provide the pumps and valves of the fluid-operated inflatable device, as described herein, are susceptible to a number of processes and risks that can lead to degradation and/or failure of the piezoelectric elements and the pumps and valves with which they are associated.

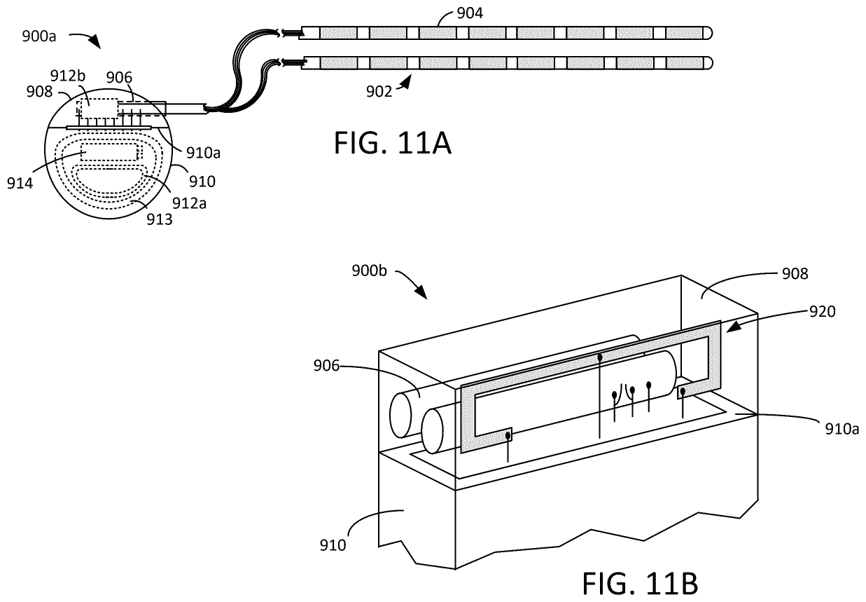

The disclosed power transmission device 150 can additionally be used to provide power to, and to charge the battery of, other types of implantable medical devices. For example, FIGS. 11A and 11B show different examples of implantable neurostimulators 900a and 900b that can be charged or powered by the disclosed power transmission device 150. Examples of these implantable neurostimulators are disclosed in U.S. patent application Ser. No. 18/658,543, filed May 8, 2024, which is incorporated herein by reference in its entirety. Implantable neurostimulators 900a and 900b may for example be used to provide Spinal Cord Stimulation (SCS) or Deep Brain Stimulation (DBS).

Implantable neurostimulator 900a comprises a biocompatible device case 910 formed of a conductive material such as titanium for example. The case 910 typically holds the circuitry and power source (e.g., a battery 914) necessary for the neurostimulator to function, although neurostimulator 900a can also be powered by the disclosed power transmission device 150 continually and therefore may lack a battery. The neurostimulator 900a is coupled to electrodes 904 via one or more electrode leads 902, such that the electrodes form an electrode array. The leads 902 connect to neurostimulator 900a using lead connectors 906, which are fixed in a non-conductive header material 908 such as a non-conductive epoxy for example. Contacts at the proximal ends of the leads 902 connect to contacts in the lead connectors 906, which are in turn connected to feedthrough wires spanning between the header 908 and the case 910, and ultimately to circuitry (e.g., a circuit board) inside the case 910. The neurostimulator 900a can be programmed to provide electrical stimulation to nervous tissue via any one or more the electrodes 904.

Neurostimulator 900a as shown in FIG. 11A may include one or more telemetry antennas 912a and/or 912b used to wirelessly transmit/receive data to/from an external communication system, such as a patient external controller or a clinician programmer. In the example shown, telemetry antenna 912a is in the case 910, and may comprise a coil, although this antenna 912a could also be located in the header 908. When configured as a coil, antenna 912a can communicate data with the external communication system via magnetic induction. Telemetry antenna 912b is in the header 908 but could also be located in the case. Telemetry antenna 912b may comprise a wire, slot, or patch antenna to receive and transmit data using far-field electromagnetic fields (e.g., at 2.4 GHZ). Antennas 912a and 912b can communicate using a protocol, such as Frequency Shift Keying or Bluetooth for example.

Neurostimulator 900a may also include a receiver member 913 for wirelessly receiving the magnetic field (power) from a power transmission device such as 150. In this example, receiver member 913 comprises a receiver coil, which receives power from the power transmission device 150 by magnetic induction. Receiver member 913 is shown within the case 910 but may also be located in the header 908. A surface 910a (or a surface portion) may exist between the case 910 and the header 908. In some examples, the receiver member 913 may be an example of the receiver member 132 of FIGS. 1A to 1F and may include any of the details discussed with reference to those figures. Neurostimulator 900a can include rectification circuitry for converting current induced in the receiver member 913 by the received power to a DC current that powers the neurostimulator directly and/or charges its battery 914. When configured as a coil, the receiver member 913 can receive power from the power transmission device 150 via a magnetic field having a relatively low frequency, such as less than 150 kHz, less than 90 kHz, or equal to or less than 80 kHz.

Neurostimulator 900b as shown in FIG. 11B discloses a different antenna structure 920 which can act as both a receiver and transmitter of data, and as a receiver member for receiving power from a power transmission device such as 150. As explained in the above-incorporated '543 Application, this antenna structure 920 preferably comprises at least one planar sheet of metal formed (e.g., by stamping or milling) into the requisite shape. Antenna structure 920 in this example is provided in the non-conductive header 908. As explained in the '543 Application, the antenna structure 920 can both communicate data via far-field electromagnetic fields (e.g., at 2.4 GHz) with an external communication system, and can receive power from a power transmission device such as 150 via magnetic induction. The power received from the power transmission device 150 can occur at a higher frequency, such as a frequency higher than 5 MHz, higher than 6 MHz, or equal to or higher than 6.78 MHz or 13.56 MHz in the Industrial, Scientific, and Medical (ISM) radio band. Use of this higher power frequency is advantageous, because it lowers heating in the case 910 by reducing the impact of eddy currents, as explained in the '543 Application. The '543 Application discloses other structural and functional examples of the antenna structure 920.

While certain features of the described implementations have been illustrated as described herein, many modifications, substitutions, changes and equivalents will now occur to those skilled in the art. It is, therefore, to be understood that the will and in and in appended claims are intended to cover all such modifications and changes as fall within the scope of the embodiments.

What is claimed is:

1. A method for aligning an external charger and an implantable medical device, the method comprising:
generating a magnetic field in a transmitter member of the external charger;
detecting a first amplitude on an inner sensor member of the external charger;
detecting a second amplitude on an outer sensor member of the external charger;
computing an alignment value based on the first amplitude and the second amplitude, the alignment value representing a degree of alignment between the transmitter member and a receiver member of the implantable medical device; and
generating an indicator based on the alignment value.

2. The method of claim 1, wherein computing the alignment value includes computing a ratio of the first amplitude and the second amplitude.

3. The method of claim 1, further comprising:
generating, by a peak detector circuit, a voltage waveform from a sense coil voltage on the inner sensor member; and
detecting, by a controller, the first amplitude from the voltage waveform.

4. The method of claim 3, further comprising:
sampling, by the controller, the voltage waveform to detect the first amplitude.

5. The method of claim 3, further comprising:
detecting the sense coil voltage using a voltage divider connected to the inner sensor member.

6. The method of claim 1, further comprising:
generating, by a peak detector circuit, a voltage waveform from a sense coil voltage on the outer sensor member; and
detecting, by a controller, the second amplitude from the voltage waveform.

7. The method of claim 6, further comprising:
sampling, by the controller, the voltage waveform to detect the second amplitude.

8. The method of claim 6, further comprising:
detecting the sense coil voltage using a voltage divider connected to the outer sensor member.

9. The method of claim 1, further comprising:
adjusting a strength of the magnetic field.

10. The method of claim 9, further comprising:
detecting a temperature of the transmitter member; and
adjusting the strength of the magnetic field based on the temperature.

11. An external charger for an implantable medical device, the external charger comprising:
a power converter configured to generate a magnetic field on a transmitter member of the external charger;
an alignment detector configured to:
detect a first amplitude on an inner sensor member of the external charger;
detect a second amplitude on an outer sensor member of the external charger; and
compute an alignment value based on the first amplitude and the second amplitude, the alignment value representing a degree of alignment between the transmitter member and a receiver member of the implantable medical device; and
an indicator generator configured to generate an indicator based on the alignment value.

12. The external charger of claim 11, wherein the alignment detector includes a peak detector circuit configured to generate a first voltage waveform from a sense coil voltage on the inner sensor member and generate a second voltage waveform from a sense coil voltage on the outer sensor member.

13. The external charger of claim 12, wherein the alignment detector includes a controller configured to detect the first amplitude from the first voltage waveform and detect the second amplitude from the second voltage waveform.

14. The external charger of claim 13, wherein the controller is configured to sample the first voltage waveform and the second voltage waveform to detect the first amplitude and the second amplitude, respectively.

15. The external charger of claim 11, wherein the alignment detector includes a voltage divider connected to the inner sensor member and the outer sensor member.

16. The external charger of claim 11, wherein the power converter includes a first switch and a second switch, the external charger further comprising:
a pulse width modulator configured to generate a control signal; and
activating the first switch and the second switch based on the control signal.

17. An apparatus comprising:
an implantable medical device including a receiver member; and
an external charger having a transmitter member, an inner sensor member, and an outer sensor member, the external charger configured to:
generate a magnetic field on the transmitter member;
detect a first amplitude on the inner sensor member;
detect a second amplitude on the outer sensor member; and
compute an alignment value based on the first amplitude and the second amplitude, the alignment value representing a degree of alignment between the transmitter member and the receiver member; and
generate an indicator based on the alignment value.

18. The apparatus of claim 17, wherein the external charger is configured to compute the alignment value as a ratio of the first amplitude and the second amplitude.

19. The apparatus of claim 17, wherein the implantable medical device includes a urology medical implant.

20. The apparatus of claim 17, wherein the external charger is configured to a strength of the magnetic field while charging the implantable medical device.
